(19) United States
(12) Patent Application Publication
(10) Pub. No.: US 2025/0018392 A1
(43) Pub. Date: Jan. 16, 2025

(54) AUTOMATED DROPLET MANIPULATION IN MICROFLUIDIC SYSTEMS

(71) Applicants: École Polytechnique Fédérale de Lausanne ( EPFL), Lausanne (CH); European Molecular Biology Laboratory, Heidelberg (DE)

(72) Inventors: Jatin PANWAR, Chavannes Pres Renens (CH); Ramesh UTHARALA, Heidelberg (DE); Christoph MERTEN, Lausanne (CH); Laura FENNELLY, Kilmacow (IE)

(21) Appl. No.: 18/714,265

(22) PCT Filed: Nov. 29, 2022
(86) PCT No.: PCT/EP2022/083721

§ 371 Date: May 29, 2024

(30) Foreign Application Priority Data
Nov. 29, 2021 (EP) 21211135.5

(51) Int. Cl. B01L 3/00

(52) U.S. Cl. CPC B01L 3/502761; B01L 3/502746; B01L 2200/0652; B01L 2300/0816; B01L 2300/0864; B01L 2400/0415; B01L 2400/0622; B01L 2400/082

(57) ABSTRACT

Disclosed herein is a method of operating a microfluidic system, a microfluidic system for manipulating sample objects, and a control system for operating a microfluidic system. The method according to the invention is for operating a microfluidic system that comprises a manipulation zone for manipulating sample objects flowing through the manipulation zone, a first detection zone arranged upstream of or in the manipulation zone and a second detection zone arranged upstream of, in or downstream of the manipulation zone. A first count is determined that characterizes a number of sample objects flowing through the first detection zone. Sample objects are manipulated in the manipulation zone. A second count is determined that characterizes a number of sample objects flowing through the second detection zone. One or more manipulation parameters for manipulating the sample objects in the manipulation zone are adjusted based on the first count and the second count.

### FIELD OF THE INVENTION

The present invention is in the field of microfluidics. In particular, the invention relates to a method of operating a microfluidic system, a microfluidic system for manipulating sample objects, and a control system for operating a microfluidic system.

### BACKGROUND

Microfluidic droplet manipulation such as droplet sorting and droplet fusion has become an integral part of many biochemical analysis methods and high-throughput screening applications, for example in drug discovery screens, see e.g. J. J. Agresti et al., Proceedings of the National Academy of Science, 107(9), 4004-4009 (2010) and L. Mazutis et al., Nature Protocols 8(5), 870-891 (2013). Monitoring the droplets by electrical or optical means allows for a partial automation of the droplet manipulation as e.g. reported in E. V. Moiseeva et al., Sensors and Actuators B 155, 408-414 (2011) and M. Sesen et al., Scientific Report 10:8736 (2020). However, despite this immense potential, typical microfluidic setups are still very complex in terms of operation and control and therefore hardly accessible to non-specialized laboratories.

A microfluidic setup for manipulating microdroplets may for example have a number of control parameters such as the flow rates of one or more fluids that need to be configured precisely by highly trained specialists to reach the desired outcome. Moreover, microfluidic setups are often very sensitive to variations in conditions, e.g. in ambient parameters such as humidity and temperature as well as control parameter such as the flow rates, which may require costly and time-consuming manual validation and adjustments to achieve long-term stability. This may limit the efficiency and throughput of the experiments.

### SUMMARY OF THE INVENTION

It is thus an object of the invention to improve the efficiency and throughput of a microfluidic system for manipulating microfluidic droplets and to simplify the handling thereof.

This object is met by a method of operating a microfluidic system according to claim 1, a microfluidic system for manipulating sample objects according to claim 15, and a control system for operating a microfluidic system according to claim 21. Embodiments of the present invention are detailed in the dependent claims.

The method of operating a microfluidic system according to the invention may be used for operating microfluidic systems that comprise a manipulation zone for manipulating sample objects flowing through the manipulation zone, a first detection zone arranged upstream of or in the manipulation zone and a second detection zone arranged upstream of, in or downstream of the manipulation zone. The method comprises (1) determining a first count N₁ that characterizes a number of sample objects flowing through the first detection zone; (2) manipulating sample objects in the manipulation zone; (3) determining a second count N₂ that characterizes a number of sample objects flowing through the second detection zone; and (4) adjusting one or more manipulation parameters for manipulating the sample objects in the manipulation zone based on the first count and the second count. The above numbering is for clarity only and does not imply a certain order of execution. As far as technically feasible, the method may be executed in an arbitrary order and steps thereof may also be executed simultaneously at least in part.

The microfluidic system may for example comprise one or more microfluidic channels that extend through the first detection zone, the manipulation zone, and the second detection zone. The microfluidic system may further comprise means for manipulating the sample objects in the manipulation zone, for example one or more manipulation electrodes and/or one or more channel junctions. In some embodiments, the microfluidic system may also comprise means for detecting sample objects in the first detection zone and/or in the second detection zone, for example one or more detection electrodes and/or one or more detection windows. In a preferred embodiment, the microfluidic system is a microfluidic system for manipulating sample objects according to any one of the embodiments described herein.

The first detection zone and/or the second detection zone may for example be zones in and/or adjacent to which one or more means for detecting sample objects are arranged, e.g. a zone around or adjacent to a respective assembly of one or more detection electrodes and/or a zone underneath a respective detection window. Additionally or alternatively, one or both of the first and second detection zones may e.g. each be a region of interest in an image of the microfluidic system or a part thereof, which may for example be defined by a user. In some embodiments, the first and second detection zones may be different regions of interest in the same image.

In some embodiments, the second detection zone may be arranged downstream of the first detection zone, wherein the first detection zone may also be referred to as an upstream detection zone and the second detection zone may also be referred to as a downstream detection zone. The first detection zone may for example be arranged upstream of the means for manipulating sample objects of the microfluidic system, e.g. upstream of or in the manipulation zone. The second detection zone may for example be arranged downstream of the means for manipulating sample objects of the microfluidic system, e.g. in or downstream of the manipulation zone. The first count N₁ may be an input count Nᵢₙ that characterizes a number of non-manipulated sample objects flowing through the first detection zone prior to manipulation in the manipulation zone. The second count N₂ may be an output count Nₒᵤₜ that characterizes a number of manipulated sample object flowing through the second detection zone after manipulation in the manipulation zone.

In other embodiments, both of the first and second detection zones may be arranged upstream of the means for manipulating sample objects of the microfluidic system, e.g. upstream of or in the manipulation zone. For example, the first and second detection zones may be arranged along different channels that merge with each other at a merging junction, e.g. as detailed below.

The sample objects may be microscopic objects, for example objects with a size or a diameter between 1 μm and 1 mm. The sample objects may e.g. be microdroplets, microbubbles, solid microparticles, single biological cells, clusters or aggregates of biological cells, or a combination thereof. The sample objects may for example be provided in a carrier fluid that flows through the microfluidic system, wherein the carrier fluid may for example be an oil, water, or an aqueous solution. In the context of this disclosure, terms such as “upstream” and “downstream” may be used relative to a flow direction of the sample objects and/or of the carrier fluid in the microfluidic system. A first element may for example be referred to as upstream (downstream) of a second element if the sample objects and/or the carrier fluid first flow past or through the first (second) element before flowing past or through the second (first) element.

The first count and/or the second count may for example characterize a number of sample objects flowing past one or more detection points or detection elements in the respective detection zone, e.g. into the respective detection zone, out of the respective detection zone, past a center of the detection zone or past a detection electrode. The first count and/or the second count may be a number of sample objects flowing through the respective detection zone, for example a total number of sample object flowing through the respective detection zone in a pre-defined time interval (e.g. N₁=100). In other embodiments, the first count and/or the second count may for example be a rate or frequency of sample objects flowing through the respective detection zone (e.g. N₁=100 s⁻¹).

The first count and/or the second count may characterize the number of all sample objects flowing through the respective detection zone. In other embodiments, the first count and/or the second count may characterize a number of a subset of sample objects, e.g. a number of sample objects having one or more pre-defined properties, for example a number of sample objects of a certain type (e.g. a certain cell type), a number of sample objects of a certain size (e.g. sample objects having a diameter within a pre-defined range), a number of sample objects of a certain shape (e.g. sample objects having an aspect ratio or ellipticity within a predefined range), a number of sample objects with a certain spacing (e.g. sample objects having an inter-object spacing within a pre-defined range), and/or a number of sample objects with a certain composition.

The first count and/or the second count may for example be determined by performing optical, acoustic and/or electrical measurements on the sample objects flowing through the respective detection zone. The measurements may e.g. be performed in the respective detection zone to detect the presence of sample objects in the respective detection zone and/or to determine one or more properties of the sample objects such as their size and/or velocity. The measurements may be performed at one or more discrete points in time and/or continuously, for example to record a time trace of a measurement signal.

An optical measurement may for example be performed by measuring an intensity of light transmitted through, reflected from, and/or emitted from the respective detection zone, e.g. fluorescence emitted by the sample objects. In some embodiments, the optical measurement may be a spatially resolved optical measurement and may for example be performed by taking a microscope image of the respective detection zone, e.g. to identify sample objects therein. In other embodiments, the optical measurement may be a point-like optical measurement and may for example be performed by measuring the intensity of light with a point-like detector such as photodiode.

An acoustic measurement may for example be performed by measuring an intensity and/or an amplitude of a sound wave transmitted through and/or reflected from the respective detection zone, wherein the sound wave may in particular be an ultrasound wave. For example, a sound wave propagating perpendicular to a direction of flow of the sample objects in the respective detection zone may be generated on one side of a channel or chamber of the microfluidic system and an intensity and/or an amplitude of the sound wave may be measured on the opposite side of the channel or chamber. In some embodiments, the sound wave may be a surface acoustic wave, e.g. a surface acoustic wave propagating along a wall of a channel or chamber of the microfluidic system.

An electrical measurement may for example be performed by measuring a current and/or a voltage through/at one or more electrical components, in particular electrodes, arranged in and/or adjacent to the respective detection zone. Preferably, the electrical measurement is an impedance measurement, e.g. a measurement for determining a direct-current (DC) resistance and/or an alternating-current (AC) impedance between one or more pairs of electrodes. Additionally or alternatively, the electrical measurement may for example also be an inductive measurement and/or a capacitive measurement, e.g. a measurement for determining an inductance and/or a capacitance of one or more electrical components.

Manipulating the sample objects in the manipulation zone may comprise any type of manipulation of the sample objects that may affect an output count Nₒᵤₜ characterizing a number of sample objects flowing through a zone downstream of the point or zone where the sample objects are manipulated, e.g. a number of sample objects flowing through the second/downstream detection zone. Manipulating the sample objects may for example comprise manipulating trajectories of the sample objects, for example such that only some of the sample objects entering the manipulation zone flow through the second detection zone. Additionally or alternatively, manipulating the sample objects may for example comprise changing the total number of sample objects, e.g. by removing or merging sample objects, for example such that a number of sample objects entering the second detection zone is different from a number of sample objects entering the manipulation zone. Additionally or alternatively, manipulating the sample objects may also comprise changing a number of sample objects having one or more pre-defined properties, for example by changing a size of the sample objects.

Manipulating the sample objects may in particular comprise applying a force to the sample objects, for example to manipulate trajectories of the sample objects, e.g. such that a given sample object flows through the second detection zone or does not flow through the second detection zone. The force may for example be generated by applying an electric field (e.g. electrophoresis and/or dielectrophoresis), a magnetic field (magnetophoresis), a soundwave (acoustophoresis), and/or an electromagnetic wave (e.g. light). Additionally or alternatively, the force may also be generated by a fluid, e.g. by generating a transverse flow of a fluid to deflect sample objects and/or by generating a hydrodynamic and/or viscoelastic force on the sample objects in a flow of a fluid. In some embodiments, the force may also be generated by the sample objects themselves, e.g. by collisions between sample objects.

Manipulating the sample objects in the manipulation zone may for example comprise deflecting or guiding the sample objects into one of a plurality of sorting channels. The sample objects may be deflected based on a sorting control signal, wherein the sorting control may for example be based on a type of the sample object, e.g. such that sample objects of a first type are deflected into a first sorting channel and sample objects of a second type are deflected into a second sorting channel. In some embodiments, the method may comprise determining the type of the sample objects, e.g. in the first detection zone and/or in the manipulation zone, and/or generating the sorting control signal based on the type of the sample objects, for example as detailed below for the control system according to the invention. The second count N₂ may characterize a number of sample objects flowing along or deflected into one of the sorting channels. The second detection zone may for example be arranged along the respective sorting channel.

Additionally or alternatively, manipulating the sample objects in the manipulation zone may also comprise merging two or more sample objects to a composite sample object. The two or more sample objects may for example be merged by applying a force to some or all of the respective sample objects, e.g. such that the two or more sample objects come in contact with each other and merge or fuse together, for example by electro-coalescence and/or by a collision between the two or more sample objects. In some embodiments, merging the two or more sample objects may comprise merging two or more fluid flows containing the sample objects, e.g. at one or more merging junctions, wherein the fluid flows may for example be merged such that sample objects contained in different fluid flows collide with each other. In some embodiments, the first detection zone may be arranged upstream of the merging junction and the second detection zone may be arranged at or downstream of the merging junction. In other embodiments, the second detection zone may also be arranged upstream of the merging junction, e.g. such that the first and second detection zones are arranged along different channels leading to the merging junction. In one example, the first and second detection zones are different regions of interest in a same image of the merging junction, e.g. regions of interest associated with different channels leading to or from the merging junction.

The manipulation of the sample objects in the manipulation zone may be characterized by one or more manipulation parameters that affect how the sample objects are manipulated. The manipulation parameters may directly pertain to the manipulation process itself, i.e. the manipulation parameters may not relate to a decision process, e.g. for deciding whether or not to perform a manipulation on a certain sample object or how to perform the manipulation on a certain sample object. For example, the manipulation parameters may not relate to a classification of sample objects, e.g. for sorting the sample objects. Instead, the manipulation parameters may for example determine the force applied to the sample objects, e.g. a direction and/or a magnitude of the force. The manipulation parameters may for example comprise one or more flow rates or flow velocities of fluid flows in the microfluidic system, one or more parameters associated with an electric field for manipulating the sample objects, one or more parameters associated with a magnetic field for manipulating sample objects, one or more parameters associated with a soundwave for manipulating the sample objects and/or one or more parameters associated with an electromagnetic wave for manipulating the sample objects. Additionally or alternatively, the manipulation parameters may also comprise one or more ambient parameters such as a temperature or a humidity.

One or more of these manipulation parameters for manipulating the sample objects in the manipulation zone are adjusted based on the first count and the second count. This may for example comprise comparing the second count N₂ to the first count N₁, in particular comparing the output count Nₒᵤₜ to the input count Nᵢₙ, and adjusting the one or more manipulation parameters based on an outcome of this comparison. For example, the ratio N₂/N₁ may be determined and compared to a reference value or reference range. The one or more manipulation parameters may for example be adjusted based on a deviation of the ratio N₂/N₁ from the reference value or reference range, e.g. by adjusting the manipulation if the ratio N₂/N₁ deviates from the reference value or is outside of the reference range. This may for example allow for implementing a feedback control for the manipulation of the sample objects, e.g. as detailed below.

Additionally or alternatively, the one or more manipulation parameters may also be adjusted based on the first count and the second count to optimize the manipulation of sample objects. This may for example comprise determining an efficiency metric η of the manipulation of the sample objects based on the first count and/or the second count and optimizing the efficiency metric by adjusting the one or more manipulation parameters. The efficiency metric may for example characterize a deviation of the second count N₂ and/or of the ratio N₂/N₁ from an optimum value or optimum range. The efficiency metric may for example be the difference to the optimum value or optimum range (e.g. an error such as a fraction of sample objects that are sorted incorrectly, for example a 2% error rate) or a fraction relative to the optimum value or optimum range (e.g. an efficiency such as a sorting efficiency or sorting purity, for example a 98% efficiency). Accordingly, optimizing the efficiency metric may for example comprise minimizing or maximizing the efficiency metric, e.g. to achieve the lowest or highest possible efficiency metric or an efficiency metric below or above a pre-defined value. The optimum value or optimum range may for example be the efficiency metric expected under ideal conditions, which may for example be obtained by validation measurements and/or based on a control signal such as the sorting control signal.

In one example, manipulating the sample objects comprises sorting sample objects of a first type and a second type into a pair of sorting channels such that objects of the first type should end up in a first sorting channel and objects of a second type should end up in a second sorting channels. The first count N₁ (input count Nᵢₙ) may for example be the number of (non-sorted) sample objects upstream of a sorting junction at which the first and second sorting channels split, the second count N₂ (output count Nₒᵤₜ) may for example be the number of (sorted) sample objects in the first channel and the efficiency metric η may for example be the ratio N₂/N₁ divided by the ratio N_{A}/N₁ with N_{A} e.g. being the number of sample objects of the first type detected in the first detection zone and/or the manipulation zone, i.e. η=N₂/N_{A}, or the ratio N_{A}/N₁ being a known fraction of the sample objects of the first type among the non-sorted sample objects.

In some embodiments, manipulating the sample objects in the manipulation zone comprises generating a force on the sample objects by applying a voltage to one or more electrodes of the microfluidic system, e.g. for sorting or merging the sample objects. The one or more electrodes may for example be arranged in or adjacent to the manipulation zone and may for example be configured to generate an electric field in a channel or a chamber of the microfluidic system in the manipulation zone. Applying the voltage to the one or more electrodes may in particular comprise applying a voltage pulse or a pulse sequence comprising a plurality of voltage pulses to the one or more electrodes. Each voltage pulse may be a DC voltage pulse of a substantially constant voltage or may be an AC voltage pulse of an oscillating voltage.

Each voltage pulse may be characterized by one or more pulse parameters such as a pulse duration P_{V}, a pulse delay L_{V} (e.g. after an event such as detecting a sample object in the first detection zone and/or in the manipulation zone or after receiving a control signal such as a sorting control signal), an amplitude A_{V} (e.g. a peak-to-peak voltage), and/or a frequency f_{V} (e.g. an oscillation frequency of an AC voltage pulse). The one or more manipulation parameters may comprise one or more of these pulse parameters, i.e. one or more of an amplitude, a frequency, a pulse duration, and a pulse delay of the voltage. Additionally or alternatively, the manipulation parameters may for example also comprise one or more pulse sequence parameters such as a number of pulses in the sequence and/or a duty cycle of the sequence. In some embodiments, the one or more manipulation parameters may additionally or alternatively comprise one or more flow rates of fluid flows in the microfluidic system, e.g. as detailed below.

In some embodiments, adjusting the one or more manipulation parameters comprises determining an efficiency metric η of the manipulation of the sample objects based on the first count and/or the second count, e.g. as detailed above, and optimizing the efficiency metric by scanning one or more manipulation parameters to determine a set of optimized manipulation parameters. Scanning a manipulation parameter may for example comprise adjusting a value of the manipulation parameter in discrete steps, e.g. from an initial value to a final value. For each set of values of the manipulation parameters, the efficiency metric may be determined, e.g. to determine a minimum or maximum value of the efficiency metric between the initial and final value and/or to determine a gradient of the efficiency metric in the parameter space spanned by the manipulation parameters. In embodiments where manipulating the sample objects comprises generating a force on the sample objects by applying a voltage, the efficiency metric may for example be optimized by scanning one or more of the amplitude A_{V}, the frequency f_{V}, the pulse duration P_{V}, and the pulse delay L_{V} of the voltage to determine a set of optimized manipulation parameters, e.g. by scanning at least the pulse delay, at least the pulse delay and the pulse duration or at least the pulse delay, the pulse duration and the amplitude. Additionally or alternatively, one or more flow rates of fluid flows in the microfluidic system may be scanned to optimize the efficiency metric.

In some embodiments, the manipulation parameters may be scanned in a pre-defined order, e.g. by first scanning a first manipulation parameter and then scanning a second manipulation parameter. The manipulation parameters may in particular be scanned iteratively in a pre-defined order, e.g. by scanning a first parameter and, for each value of the first parameter, scanning a second parameter while keeping the first parameter constant (i.e. performing a two-dimensional raster scan). In other words, the manipulation parameters may be scanned using a plurality of nested iteration loops (e.g. for-loops) for scanning a respective one of the manipulation parameters, e.g. a first iteration loop for the first parameter (first level iteration loop) and a second iteration loop for the second parameter, wherein the second iteration loop is executed during each iteration of the first iteration loop (second level iteration loop). This may be generalized to an arbitrary number of manipulation parameters, e.g. three, four or five manipulation parameters, i.e. performing a three-, four- or five-dimensional raster scan in parameter space.

In embodiments where manipulating the sample objects comprises generating a force on the sample objects by applying a voltage, optimizing the efficiency metric may for example comprise scanning two or more of the amplitude A_{V}, the frequency f_{V}, the pulse duration P_{V}, and the pulse delay L_{V} of the voltage iteratively in a pre-defined order while the remaining parameters are kept at constant values. For example, the pulse delay and the pulse duration may be scanned using a pair of nested iteration loops, e.g. a first level iteration loop for the pulse duration and a second level iteration loop for the pulse delay. In other words, the pulse duration may be scanned at constant amplitude and frequency, wherein for each value of the pulse duration the pulse delay is scanned at constant pulse duration.

In some embodiments, an optimization criterion ηₑ may be defined for terminating the scan of the one or more manipulation parameters. The manipulation parameters may for example be scanned until the efficiency metric meets the optimization criterion. The optimization criterion may for example be a lower bound or an upper bound for the efficiency metric that is to be achieved, wherein the scan may e.g. be terminated when the efficiency metric exceeds the lower bound (e.g. is larger than a minimum manipulation efficiency that is to be achieved) or is below the upper bound (e.g. is smaller than a maximum manipulation error that is to be achieved). Additionally or alternatively, the optimization criterion may also define a condition that is to be met by a different quantity, for example a stability metric as detailed below.

In embodiments where manipulating the sample objects comprises generating a force on the sample objects by applying a voltage, optimizing the efficiency metric may for example comprise scanning the pulse delay L_{V} at a constant pulse duration P_{V} and a constant amplitude A_{V} until the efficiency metric η meets an optimization criterion ηₑ. If the optimization criterion ηₑ cannot be met by scanning the pulse delay L_{V} at the constant pulse duration P_{V} and the constant amplitude A_{V}, e.g. by scanning the pulse delay all the way from an initial value to a final value, the pulse duration may be scanned at the constant amplitude A_{V}. In some embodiments, this may be implemented as a pair of nested iteration loops as described above, i.e. for each value of the pulse duration, the pulse delay may be scanned again and, if the optimization criterion still cannot be met by scanning the pulse delay, the pulse duration may be incremented and so on (two-dimensional raster scan).

In some embodiments, optimizing the efficiency metric additionally or alternatively comprises scanning the amplitude A_{V} if the optimization criterion ηₑ cannot be met by scanning the pulse duration P_{V} at the constant amplitude A_{V}. For example, the amplitude may be scanned in a first level iteration loop, the pulse duration in a second level iteration loop executed in each iteration of the first level iteration loop, and the pulse delay in a third level iteration loop executed in each iteration of the second level iteration loop (three-dimensional raster scan). In some embodiments, optimizing the efficiency metric additionally or alternatively comprises scanning one or more other manipulation parameter, for example the frequency f_{V} and/or an input rate of the sample objects as detailed below if the optimization criterion ηₑ cannot be met by scanning the amplitude A_{V}.

Additionally or alternatively, more sophisticated optimization methods may be employed for optimizing the efficiency metric, for example a gradient-based optimization such as gradient descent and/or a Hessian-based optimization method such as Newton's method. Additionally or alternatively, optimizing the efficiency metric may also comprise a sampling of one or more manipulation parameters, in particular random sampling of one or more manipulation parameters, e.g. using a Monte-Carlo method.

In some embodiments, the sample objects flow into the manipulation zone at a constant input rate f_{D}, wherein the input rate f_{D} may for example quantify the number of sample objects entering the manipulation zone per second. The sample objects may flow into the manipulation zone one after the other with a substantially constant time difference between subsequent sample objects. For example, the sample objects may have a substantially constant velocity and a substantially constant spacing between subsequent sample objects. The method may comprise optimizing the efficiency metric η at two or more input rates f_{D} to determine respective sets of optimized manipulation parameters, e.g. a first set of optimized manipulation parameters (A_{V}, f_{V}, P_{V}, L_{V}) at a first input rate and a second set of optimized manipulation parameters (A_{V}, f_{V}, P_{V}, L_{V}) at a second input rate. In some embodiments, the method may also comprise scanning the input rate and/or one or more manipulation parameters determining the input rate to optimize the efficiency metric.

In some embodiments, the optimization criterion ηₑ is a minimum manipulation efficiency, i.e. lower bound for the efficiency metric that is to be achieved, or a maximum manipulation error, i.e. upper bound for the efficiency metric that is to be achieved, and the method further comprises determining a maximum input rate at which the minimum manipulation efficiency or maximum manipulation error, respectively, can be achieved. This may for example comprise iteratively increasing the input rate and optimizing one or more manipulation parameters, e.g. one or more of the amplitude A_{V}, the frequency f_{V}, the pulse duration P_{V}, and the pulse delay L_{V} of the voltage, at each value of the input rate, until an input rate is reached at which it is no longer possible to meet the optimization criterion by optimizing the respective manipulation parameters.

In some embodiments, the method further comprises determining a stability metric for the set of optimized manipulation parameters. The stability metric may for example characterize how stable the efficiency metric is over time for the set of optimized manipulation parameters, e.g. how much the efficiency metric deteriorates over time and/or how much the efficiency metric fluctuates overtime. Determining the stability metric may comprise determining the efficiency metric η for the set of optimized manipulation parameters again at one or more later points in time, e.g. to record a time trace of the efficiency metric. The stability metric may for example quantify or be a time derivative of the efficiency metric and/or a variance of the efficiency metric. In some embodiments, the method may also comprise optimizing the stability metric, e.g. to determine a set of optimized manipulation parameters for which the stability metric exhibits an optimum (e.g. a maximum of the stability metric, which may e.g. correspond to a minimum of the time derivative and/or of the variance), or meets a pre-defined stability criterion. In one example, the method comprises determining a maximum input rate at which a pre-defined minimum manipulation efficiency as the optimization criterion and a pre-defined minimum stability as the stability criterion may be achieved at the same time.

In some embodiments, a set of optimized manipulation parameters determined by optimizing one or more of the efficiency metric, the stability metric, and the input rate may be used for a subsequent operation of the microfluidic system, e.g. to manipulate sample objects for performing an experiment such as a drug discovery screen. The determination of the set of optimized manipulation parameters may also be referred to as optimization or training mode in the following, whereas the subsequent (normal) operation of the microfluidic system may also be referred to as screening mode. In other words, the method may comprise determining a set of optimized manipulation parameters in training mode and subsequently operating the microfluidic system in screening mode using the set of optimized manipulation parameters.

The method may further comprise monitoring the first count and the second count continuously during operation of the microfluidic system, e.g. by repeatedly determining the first count and the second count while manipulating sample objects in screening mode. The first and second counts may for example be used as inputs for a feedback control of one or more manipulation parameters, e.g. by continuously adjusting the one or more manipulation parameters based on the first count and the second count. This may for example comprise determining the efficiency metric based on the first count and/or the second count, comparing the efficiency metric to a set point or set point range, and adjusting the one or more manipulation parameters if the efficiency metric deviates from the set point or set point range. In some examples, this may comprise optimizing the efficiency metric again if the efficiency metric deviates from the set point or set point range, e.g. as detailed above.

Additionally or alternatively, the first and second counts may also be used for determining whether a failure has occurred during operation of the microfluidic system. A failure may for example be caused by clogging of a channel of the microfluidic system, failure of a pump or a valve of the microfluidic system, or a reservoir of the microfluidic system running empty. A failure may be associated with a change or fluctuation of the first count, the second count, and/or the efficiency metric, in particular a sudden change or fluctuation. A failure may for example be detected if the first count, the second count, and/or the efficiency metric drops below or exceeds a respective pre-defined threshold and/or if a magnitude of a derivative of the respective quantity exceeds a pre-defined threshold. In case of a failure being detected, the method may for example comprise temporarily pausing or indefinitely terminating operation of the microfluidic system. Operation of the microfluidic system may for example be paused for a pre-defined settle time. After the pre-defined settle time, operation of the microfluidic system may be resumed and/or the first count, the second count, and/or the efficiency metric may be determined again, e.g. to determine whether the failure persists. If the failure persists, operation of the microfluidic system may again be paused for the pre-defined settle time. This procedure may for example be repeated a pre-defined number of times, e.g. three times, and if the failure still persists, operation of the microfluidic system may be terminated, e.g. until a user resets the system.

In a preferred embodiment, the sample objects are microdroplets forming a dispersed phase in a continuous phase. A size or a diameter of the microdroplets may for example be between 1 μm and 1 mm, in some examples between 5 μm and 200 μm. The microdroplets may for example comprise or consist of water or an aqueous solution and the continuous phase may comprise or consist of an oil or vice-versa. In some embodiments, the microdroplets may also comprise objects or substances other than the dispersed phase, for example one or more cells and/or one or more reactants for a chemical reaction.

The method may further comprises adjusting one or more flow rates of fluid flows in the microfluidic system, for example an input flow rate and/or a withdrawal rate of a fluid, e.g. of a carrier fluid that the sample objects are contained in. The method may in particular comprise adjusting one or more of an input flow rate of the dispersed phase, an input flow rate of the continuous phase, and a withdrawal rate of dispersed and continuous phases. The aforementioned parameters may for example be adjusted to obtain a certain input rate and/or flow velocity at which the microdroplets flow into the manipulation zone. Additionally or alternatively, the aforementioned parameters may also be adjusted to control a different property of the microdroplets, for example a size or a composition of the microdroplets. In some examples, one or more of the aforementioned parameters may be adjusted based on the first count and the second count, in particular in embodiments in which the sample objects are manipulated by a hydrodynamic and/or viscoelastic force. In other words, the respective parameters may be used as manipulation parameters, which may e.g. be adjusted as detailed above, for example to optimize the efficiency metric.

In some embodiments, the method may further comprise determining one or more of a flow rate (e.g. an input rate), a spacing (e.g. a distance between adjacent sample objects), a size (e.g. a diameter), and a velocity of the sample objects in the first detection zone and/or in the second detection zone. The aforementioned quantities may in particular be determined based on the electrical and/or optical measurements that are also used for determining the first count and/or the second count. The flow rate and/or the spacing may for example be determined by determining a time difference between subsequent sample objects, e.g. in combination with a known flow velocity of a carrier fluid for the sample objects. The velocity and/or the size may for example be determined by determining a duration or width of a feature or signature (e.g. a peak or dip) in a measured signal (e.g. a time trace of a voltage or light intensity) that is associated with a sample object, e.g. in combination with a known size of the sample objects and a known flow velocity of the carrier fluid, respectively. Additionally or alternatively, the size may for example be determined by determining an amplitude or height of the feature. In some embodiments, the method may also comprise determining a shape of the sample objects in the first detection zone and/or in the second detection zone, e.g. based on a shape of the feature in the measured signal.

By determining a first count characterizing a number of sample objects flowing through the first detection zone, e.g. an input count of non-manipulated sample objects, and determining a second count characterizing a number of sample objects flowing through the second detection zone, e.g. an output count of manipulated sample objects, i.e. by observing the sample objects at two different points in the microfluidic system, the method according to the present invention allows for real-time monitoring and validation of the manipulation of the sample objects in the manipulation zone. For example, an efficiency of the manipulation may be monitored continuously during operation of the microfluidic system by observing the sample objects prior to and after performing the manipulation. By adjusting one or more manipulation parameters associated with the manipulation of the sample objects based on the first count and the second count, the present invention for example enables an optimization to improve the efficiency and throughput of the manipulation and/or a feedback control of the manipulation to improve the robustness of the manipulation with regard to variations in conditions such as in ambient parameters. Moreover, the method can be automated easily to provide automated monitoring, optimization, and/or feedback control. Thereby, the present invention simplifies the handling of microfluidic systems for manipulating sample objects such as microdroplets and improves the efficiency and throughput of the manipulation.

The present invention further provides a microfluidic system for manipulating sample objects. The microfluidic system according to the invention comprises a microfluidic channel extending through a manipulation zone, a first detection zone that is arranged upstream of or in the manipulation zone, and a second detection zone that is arranged upstream of, in or downstream of the manipulation zone. The microfluidic system comprises a first electrode assembly arranged in the first detection zone for performing electrical measurements on sample objects within the microfluidic channel and a second electrode assembly arranged in the second detection zone for performing electrical measurements on sample objects within the microfluidic channel. The microfluidic system further comprises means for manipulating sample objects in the manipulation zone.

The microfluidic system may for example be operated using a method of operating a microfluidic system according to any one of the embodiments described herein and/or using a control system for operating a microfluidic system according to any one of the embodiments described herein. In particular, the first electrode assembly may be used for determining a first count that characterizes a number of sample objects flowing through the first detection zone, the means for manipulating sample objects in the manipulation zone may be used for manipulating sample objects in the manipulation zone, and the second electrode assembly may be used for determining a second count that characterizes a number of sample objects flowing through the second detection zone. The means for manipulating sample objects in the manipulation zone may be associated with one or more manipulation parameters, which may be adjusted based on the first count and the second count.

In some embodiments, the second detection zone may be arranged downstream of the first detection zone. Additionally or alternatively, the second detection zone may be arranged downstream of the means for manipulating sample objects in the manipulation zone, e.g. in or downstream of the manipulation zone. The first detection zone, on the other hand, may for example be arranged upstream of the means for manipulating sample objects of the microfluidic system, e.g. upstream of or in the manipulation zone. In other embodiments, both of the first and second detection zones may be arranged upstream of the means for manipulating sample objects of the microfluidic system, e.g. upstream of or in the manipulation zone. For example, the first and second detection zones may be arranged along different sub-channels of the microfluidic channel that merge with each other at a merging junction.

Each of the first electrode assembly and the second electrode assembly comprises at least one electrode, which may for example be arranged in or adjacent to the microfluidic channel, e.g. such that the respective electrode is in electrical contact with an inner volume of the microfluidic channel. The electrodes may for example be arranged in and/or on one or more walls of the microfluidic channel, e.g. a bottom wall, a side wall and/or a top wall. Preferably, the first electrode assembly and/or the second electrode assembly each comprise at least two electrodes, in some examples at least three electrodes, all of which may be in electrical contact with the inner volume of the microfluidic channel. This may for example allow for performing differential electrical measurements. In some embodiments, some or all of the electrodes of a given electrode assembly may be arranged on and/or in the same wall of the microfluidic channel, e.g. the bottom wall. Additionally or alternatively, some or all of the electrodes of a given electrode assembly may be arranged on and/or in different walls of the microfluidic channel, for example opposing side walls. The microfluidic system may further comprise a respective connector or contact pad for each of the electrodes, e.g. such that each of the electrodes can be contacted individually.

The microfluidic system may comprise a substrate, in and/or on which the microfluidic channel, the first electrode assembly, the second electrode assembly, and/or the means for manipulating the sample objects are arranged. The substrate may comprise or consist of one or more layers, some or all of which may e.g. comprise or consist of an electrically insulating and/or optically transparent material. The substrate may for example comprise a channel substrate or layer, in and/or on which the microfluidic channel is formed. The substrate may also comprise an electrode substrate or layer, wherein the first electrode assembly and/or the second electrode assembly may be arranged on and/or in the electrode substrate. The electrode substrate may be bonded to the channel substrate, e.g. glued to the channel substrate and/or attached to the channel substrate by one or more mechanical fasteners such as clips or screws. The first electrode assembly and/or the second electrode assembly may be arranged on and/or in a surface of the electrode substrate facing the channel substrate, e.g. such that electrodes of the respective electrode assembly are exposed to an inner volume of the microfluidic channel when the electrode substrate is bonded to the channel substrate. Some or all of the means for manipulating the sample objects may also be arranged in and/or on the channel substrate and/or the electrode substrate. Additionally or alternatively, some or all of the means for manipulating the sample objects may also be arranged in and/or on one or more different layers of the substrate, e.g. in a cover plate or layer of the substrate.

The means for manipulating the sample objects in the manipulation zone may be configured to manipulate or to be used for manipulating the sample objects in the manipulation zone as detailed above for the method according to the invention, i.e. to manipulate the sample objects in a way that may affect an output count Nₒᵤₜ characterizing a number of sample objects flowing through a zone downstream of the means for manipulating the sample objects, e.g. a number of sample objects flowing through the second/downstream detection zone. The means for manipulating the sample objects may in particular be configured to apply a force to the sample objects, e.g. to manipulate trajectories of the sample objects. The means for manipulating the sample objects may for example be configured to generate an electric field and/or a magnetic field, to transmit and/or reflect a soundwave and/or an electromagnetic wave, and/or to generate or modify a flow of a fluid.

The means for manipulating the sample objects in the manipulation zone may for example comprise a manipulation electrode assembly for manipulating the sample objects by electrophoresis, dielectrophoresis, and/or magnetophoresis. The manipulation electrode assemble may for example comprise one or more electrodes and/or one or more inductive elements for generating an electric field and/or a magnetic field in the manipulation zone.

Additionally or alternatively, the means for manipulating the sample objects in the manipulation zone may also comprise an acoustic resonator for manipulating the sample objects by acoustophoresis. The acoustic resonator may for example support one or more resonant modes associated with a standing acoustic wave, which may for example extend across the microfluidic channel.

Additionally or alternatively, the means for manipulating the sample objects in the manipulation zone may also comprise a valve for manipulating the sample objects by adjusting a direction of flow and/or a flow velocity in the microfluidic channel. The valve may for example be configured to open and close a lateral channel that intersects with the microfluidic channel, e.g. to generate a transverse flow which may deflect sample objects flowing along the microfluidic channel. In other examples, the valve may for example be configured to partially open and close the microfluidic channel, e.g. to adjust a flow velocity in the microfluidic channel in order to generate and/or adjust a hydrodynamic and/or viscoelastic force on the sample objects in the manipulation zone.

Additionally or alternatively, the means for manipulating the sample objects in the manipulation zone may also comprise an illumination window for manipulating the sample objects by light-induced forces. The illumination window may for example be configured to transmit light of one or more wavelengths, e.g. for generating an optical potential for the sample objects such as optical tweezers. The illumination window may further be configured to withstand the light intensities required for generating a sufficiently strong optical potential. A damage threshold of the illumination window may for example be larger than 0.1 kW/cm², in some examples larger than 1 kW/cm², in one example larger than 10 kW/cm². The illumination window may further be configured for use in microscopic imaging. A transmitted wavefront error of the illumination window may for example be smaller than λ/2, in some examples smaller than λ/4, in one example smaller than λ/8 (e.g. at λ=633 nm).

Additionally or alternatively, the means for manipulating the sample objects in the manipulation zone may also comprise one or more channel junctions at which the microfluidic channel splits into a plurality of sub-channels of the microfluidic channel and/or at which a plurality of sub-channels of the microfluidic channel merge with each other.

The means for manipulating the sample objects may for example comprise a sorting junction at which the microfluidic channel splits into a plurality of sorting channels, e.g. a pair of sorting channels. A manipulation electrode assembly may for example be arranged in close vicinity to the sorting junction, e.g. to selectively deflect sample objects into one of the sorting channels by applying a voltage to the manipulation electrode assembly. The second electrode assembly may be arranged along one of the sorting channels, e.g. to determine an output count (second count) characterizing a number of sample objects that end up in the respective sorting channel.

Additionally or alternatively, the means for manipulating the sample objects may for example also comprise a merging junction at which a plurality of sub-channels of the microfluidic channel, e.g. a pair of sub-channels, merge with each other. The first electrode assembly may e.g. be arranged along one of the sub-channels of the microfluidic channel, for example between an inlet of the respective sub-channel and the merging junction, e.g. to determine an input count (first count) characterizing a number of sample objects entering the merging junction through the respective sub-channel of the microfluidic channel. The second electrode assembly may for example be arranged at or downstream of the merging junction, in some examples also downstream of additional means for manipulating the sample objects such as a manipulation electrode assembly, e.g. to determine an output count (second count) characterizing a number of composite sample objects after merging. In other examples, the second electrode assembly may be arranged along a different one of the sub-channels of the microfluidic channel than the first electrode assembly, for example between an inlet of the respective sub-channel and the merging junction, e.g. to determine a second input count (second count) characterizing a number of sample objects entering the merging junction through a second sub-channel of the microfluidic channel.

In other embodiments, the first electrode assembly may e.g. be arranged along the microfluidic channel between the merging junction and the second electrode assembly. The first electrode assembly may for example be arranged between the merging junction and a manipulation electrode assembly to determine an input count characterizing a number of sample objects flowing towards the manipulation electrode assembly. The second electrode assembly may for example be arranged downstream of the manipulation electrode assembly.

The invention further provides a control system for operating a microfluidic system that comprises a manipulation zone for manipulating sample objects flowing through the manipulation zone, a first detection zone arranged upstream of or in the manipulation zone and a second detection zone arranged upstream of, in or downstream of the manipulation zone. The control system according to the invention comprises a mount that is configured to receive the microfluidic system. The control system also comprises a measurement unit that is configured to perform optical, acoustic and/or electrical measurements in the first detection zone and in the second detection zone for detecting sample objects in the respective detection zone. The control system further comprises a manipulation unit that is configured to apply a force on sample objects in the manipulation zone for manipulating the sample objects. The control system also comprises a controller that is configured to determine a first count N₁ characterizing a number of sample objects flowing through the first detection zone and a second count N₂ characterizing a number of sample objects flowing through the second detection zone based on the optical, acoustic and/or electrical measurements performed by the measurement unit. The controller is further configured to adjust one or more manipulation parameters associated with the force applied by the manipulation unit on the sample objects in the manipulation zone based on the first count N₁ and the second count N₂.

The control system may be configured for use with a microfluidic system according to any one of the embodiments described herein. In some embodiments, the control system may also comprise one or more microfluidic systems according to any one of the embodiments described herein. In some embodiments, the control system may be configured to execute a method of operating a microfluidic system according to any one of the embodiments described herein at least in part, e.g. as detailed below.

The mount may for example be configured to hold or support the microfluidic system. The mount may further be configured to provide fluid and/or electrical connections to the microfluidic system, e.g. to one or more inlets and/or outlets of the microfluidic system and/or to one or more electrodes of the microfluidic system. In some embodiments, the mount may also be configured to translate and/or rotate the microfluidic system, e.g. to align the microfluidic system relative to the measurement unit or an imaging system.

The measurement unit may for example comprise an ammeter and/or a voltmeter that is configured to measure a current and a voltage, respectively, through/at one or more electrodes of microfluidic system, in particular electrodes of the first electrode assembly and/or the second electrode assembly. The measurement unit may comprise an amplifier such as a transimpedance amplifier, a differential amplifier, a lock-in amplifier, and/or an operational amplifier, e.g. to amplify the electric signal that is to be measured, to obtain a differential electric signal, and/or to convert a current to a voltage or vice versa. In some embodiments, the measurement unit may also comprise a processing device such as a micro-processor, a microcontroller, and/or a field-programmable gate array (FPGA) that is configured to measure the current and/or the voltage, e.g. the current and/or the voltage generated by the amplifier. The measurement unit may further comprise a current source and/or a voltage source that is configured to apply a current and a voltage, respectively, through/to one or more electrodes of microfluidic system, in particular electrodes of the first electrode assembly and/or the second electrode assembly.

Additionally or alternatively, the measurement unit may for example comprise one or more photodetectors that are configured to measure a light intensity, e.g. an intensity of light incident from a respective one of the detection zones. Each of the one or more photodetectors may for example comprise one or more photodiodes, one or more photomultiplier tubes, and/or one or more photosensitive chips such as a CCD or CMOS chip. In some embodiments, the measurement unit may also comprise one or more light sources, for example a laser or a light-emitting diode, e.g. to illuminate sample objects in one or both of the detection zones.

Additionally or alternatively, the measurement unit may for example comprise one or more acoustic detectors that are configured to measure an intensity and/or an amplitude of a sound wave, e.g. one or more piezoelectric elements and/or one or more capacitive and/or inductive microelectromechanical systems (MEMS). In some embodiments, the measurement unit may also comprise one or more sound sources, for example an ultrasonic transmitter and/or one or more piezoelectric elements.

The manipulation unit may for example be configured to apply a force on the sample objects in the manipulation zone as detailed above for the method of operating a microfluidic system. The manipulation unit may in particular be configured to generate the force by applying an electric field, a magnetic field, a soundwave, and/or an electromagnetic wave. Additionally or alternatively, the manipulation unit may also be configured to generate the force via a fluid, e.g. by generating a transverse flow of a fluid to deflect sample objects and/or by generating a hydrodynamic and/or viscoelastic force in a flow of a fluid. In some embodiments, the manipulation unit may be configured to control one or more of the means for manipulating sample objects of a microfluidic system according to any one of the embodiments described herein. The manipulation unit may for example be configured to apply a voltage and/or a current to a manipulation electrode assembly, to couple a soundwave into an acoustic resonator, to control a valve for manipulating sample objects, and/or to illuminate an illumination window with light. For this, the manipulation unit may comprise one or more of a voltage source, a current source, a sound source, and a light source.

The controller may be implemented in hardware, software or a combination thereof. The controller may for example comprise a processing device and a storage medium storing instructions for execution by the processing device to provide the functionality described herein. In some embodiments, the measurement unit and/or the manipulation unit or parts thereof may be integrated into the controller, i.e. the controller may be configured to provide some or all of the functionality of the measurement unit and/or the manipulation unit. In a preferred embodiment, the controller is configured to execute some or all of the steps of a method of operating a microfluidic system according to any one of the embodiments described herein.

The controller may be configured to determine the first count and the second count as detailed above for the method according to the invention. The controller may for example be configured to read out one or more measurement signals from the measurement unit, e.g. a measurement signal associated with the first detection zone and a measurement signal associated with the second detection zone, and to analyze the one or more measurement signals. The controller may be configured to detect the presence of a sample object in one of the detection zones by identifying a corresponding feature or signature such as a peak or dip in the respective measurement signal. For this, the controller may for example be configured to compare the measurement signal to one or more thresholds. The controller may be configured to determine the first count and the second count based on the identified features, e.g. based on a number of identified features. The controller may further be configured to determine one or more other quantities associated with the sample objects in the first detection zone and/or in the second detection zone, for example a flow rate, a spacing, a size and/or a velocity of the sample objects in the first detection zone and/or in the second detection zone.

In some embodiments, the second detection zone is arranged downstream of the first detection zone, e.g. as detailed above. The first count N₁ may be an input count Nᵢₙ that characterizes a number of non-manipulated sample objects flowing through the first detection zone prior to manipulation in the manipulation zone. The second count N₂ may be an output count Nₒᵤₜ that characterizes a number of manipulated sample objects flowing through the second detection zone after manipulation in the manipulation zone. In other embodiments, the first and second detection zones may be arranged differently, e.g. as detailed above.

The controller may be configured to adjust the one or more manipulation parameters based on the first count and the second count as detailed above for the method according to the invention. The controller may for example be configured to determine an efficiency metric based on the first count and/or the second count, e.g. as detailed above. The controller may also be configured to optimize the efficiency metric by scanning one or more manipulation parameters, e.g. as detailed above. The controller may also be configured to continuously monitor the first count and the second count during operation of the microfluidic system and to use the first and second counts as inputs for a feedback control of the one or more manipulation parameters and/or for determining whether a failure has occurred during operation of the microfluidic system, e.g. as detailed above.

In some embodiments, the manipulation unit is configured to apply the force by applying a voltage to an electrode in the manipulation zone, e.g. to an electrode of a manipulation electrode assembly. The manipulation unit may in particular be configured to apply a voltage pulse or a pulse sequence to the electrode, e.g. as detailed above. The one or more manipulation parameters may for example comprise one or more of an amplitude A_{V}, a frequency f_{V}, a pulse duration P_{V}, and a pulse delay L_{V} of the voltage, e.g. as detailed above. Additionally or alternatively, the one or more manipulation parameters may comprise one or more flow rates of fluid flows in the microfluidic system.

In some embodiments, the measurement unit is configured to perform differential electrical measurements between two or more pairs of electrodes in each of the first detection zone and the second detection zone, e.g. for detecting sample objects in the respective detection zone. In each of the detection zones, the measurement unit may for example be configured to perform differential electrical measurements between a first pair of electrodes comprising a first electrode and a reference electrode and a second pair of electrode comprising a second electrode and the reference electrode. This may for example increase the probability of detecting a sample objects and/or allow for a higher accuracy in determining properties of the sample objects such as their size.

In some embodiments, the control system further comprises a sorting unit for sorting the sample objects, e.g. to separate sample objects of a first type from sample objects of a second type. The sorting unit may for example be configured to perform optical, acoustic and/or electrical measurements on sample objects in the microfluidic system, e.g. in the manipulation zone and/or in the first detection zone, for example using the measurement unit. The sorting unit may further be configured to classify the sample objects based on the optical, acoustic and/or electrical measurements. The sorting unit may for example be configured to assign each of the sample objects to one of a plurality of classes or types, e.g. based on properties extracted from the optical, acoustic and/or electrical measurements such a size of the sample objects and/or a composition of the sample objects. The sorting unit may in particular be configured to classify the sample objects based on the presence or absence of a marker or label such as a fluorescent marker (e.g. for fluorescence-activated sorting) and/or based on the presence or absence of certain objects or elements in the sample objects such as a cell. The sorting unit may be configured to, using the manipulation unit, guide the sample objects into one of a plurality of sorting channels in the manipulation zone based on the classification, e.g. by selectively applying a force on the respective sample object based on whether or not the sample object belongs to a certain class or type. The sorting unit may for example generate a corresponding sorting control signal for the manipulation unit.

In some examples, some or all of the measurements for sorting the sample objects may be also be used for detecting sample objects in the first detection zone or vice-versa. In other words, the same measurement may be used for detecting the presence of a sample object in the first detection zone and for classifying the respective sample object. The measurement unit may for example be configured to supply the measurement signals to both the sorting unit and to the controller. In some embodiments, the measurement unit and the sorting unit may both be integrated into the controller at least in part.

In some embodiments, the manipulation unit is configured to manipulate the sample objects by merging two or more sample objects to a composite sample object, e.g. as detailed above. The manipulation unit may for example be configured to apply the force on the sample objects such that two or more sample objects collide or come in contact with each other and merge, e.g. by applying a voltage to induce electro-coalescence. Additionally or alternatively, the manipulation unit may be configured to control one or more flow rates, e.g. in sub-channels intersecting at a merging junction, for merging the two or more sample objects. The manipulation unit may for example be configured to adjust the one or more flow rates such that sample objects from different sub-channels arrive at the merging junction at the same time, e.g. such that the respective sample objects collide at the merging junction or come in contact with or close to each other at the merging junction.

### LIST OF FIGURES

In the following, a detailed description of the invention and exemplary embodiments thereof is given with reference to the figures. The figures show schematic illustrations of

FIG. 1: a microfluidic system for manipulating sample objects according to an exemplary embodiment of the invention in top view;

FIG. 2: a microfluidic system for manipulating sample objects comprising a droplet generator in accordance with an exemplary embodiment of the invention in top view;

FIG. 3: a microfluidic system for manipulating sample objects comprising a merging junction according to an exemplary embodiment of the invention in top view;

FIG. 4: a control system for operating a microfluidic system in accordance with an exemplary embodiment in side view;

FIG. 5: a flow chart of a method of operating a microfluidic system in accordance with an exemplary embodiment of the invention;

FIG. 6: examples of measurement signals for detecting sample objects obtained experimentally using a microfluidic system according to an exemplary embodiment of the invention;

FIG. 7: examples of time traces of a first/input count, a second/output count, and an efficiency metric determined in scanning mode using a microfluidic system according to an exemplary embodiment of the invention;

FIG. 8: a flow chart of a method of optimizing an efficiency metric in accordance with an exemplary embodiment of the invention; and

FIGS. 9a to 9e: screening results for droplet sorting using a method and a microfluidic system according to exemplary embodiments of the invention.

### DESCRIPTION OF THE PREFERRED EMBODIMENTS

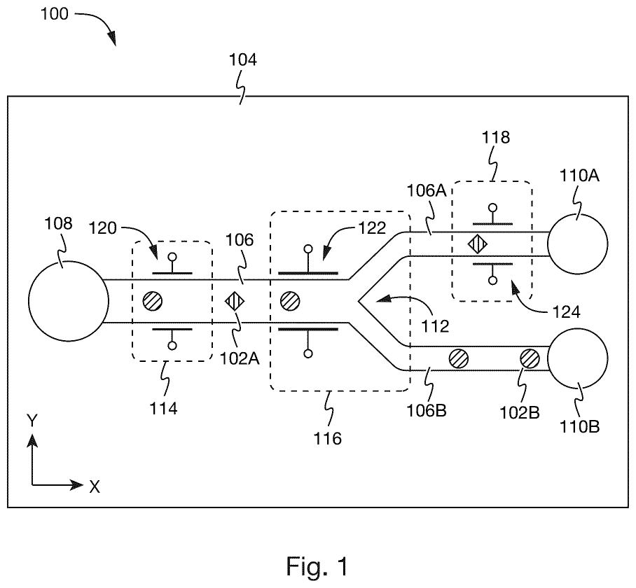

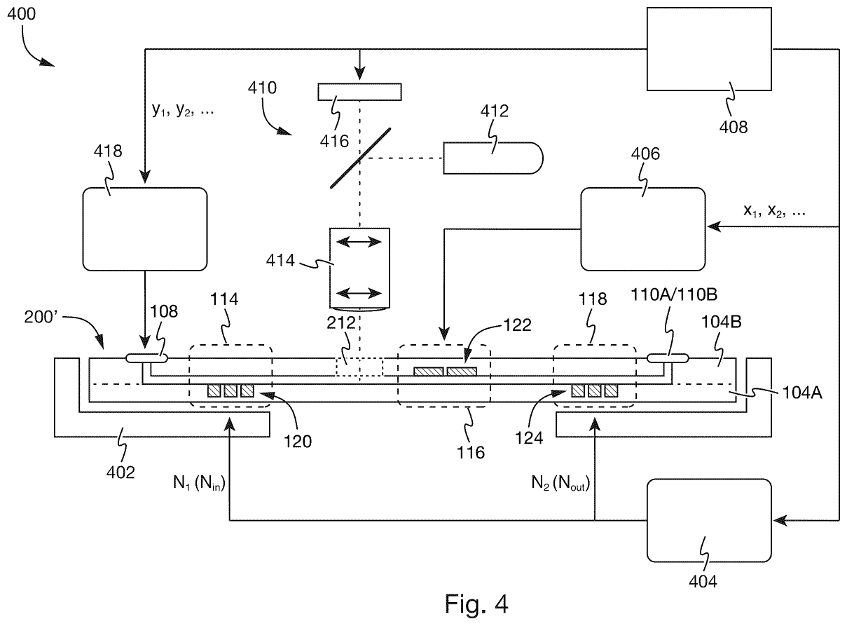

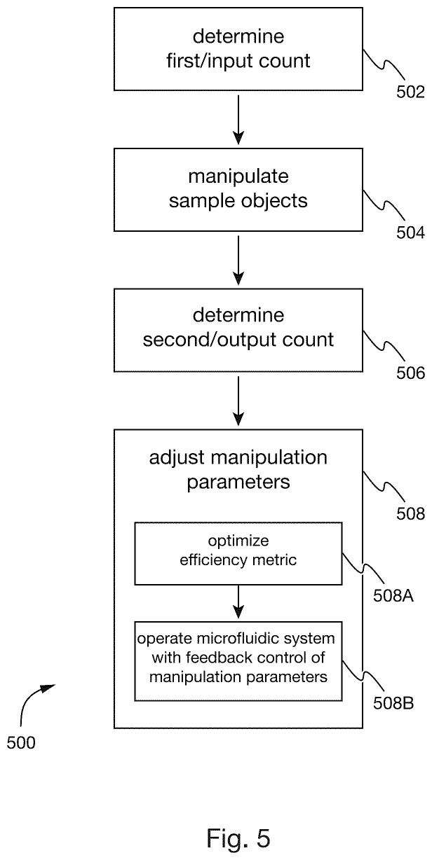

FIG. 1 depicts a schematic illustration (not to scale) of a microfluidic system 100 for manipulating sample objects 102A, 102B according to an exemplary embodiment of the invention in top view. The microfluidic system 100 may for example be operated using a method of operating a microfluidic system according to any one of the embodiments described herein, for example the method 500 of FIG. 5, and/or using a control system for operating a microfluidic system according to any one of the embodiments described herein, for example the control system 400 of FIG. 4.

The sample objects 102A, which are indicated by hatched circles in FIG. 1, are sample objects of a first type, for example microdroplets that contain a cell or a first substance. The sample objects 102B, which are indicated by hatched diamonds in FIG. 1, are sample objects of a second type, for example microdroplets that do not contain a cell or the first substance or microdroplets that contain a cell of a different type or a second substance different from the first substance. In the example of FIG. 1, the microfluidic system 100 is used for sorting the sample objects 102A, 102B to separate sample objects 102A of the first type from sample objects 102B of the second type.

The microfluidic system 100 comprises a substrate 104, which may for example comprise or consist of one or more layers (not shown). The substrate 104 and/or layers thereof may for example be formed of an electrically insulating and optically transparent substance such as glass or a transparent thermoplastic, e.g. polymethyl methacrylate (PMMA) or Polydimethylsiloxane (PDMS). A microfluidic channel 106 is arranged in or on the substrate 104. The microfluidic channel 106 may for example be fully enclosed by the substrate 104, i.e. such that the microfluidic channel 106 is sealed off from the environment of the microfluidic system 100. In other embodiments, the microfluidic channel 106 may for example be formed in a top surface of the substrate 104 and may be in contact with the environment of the microfluidic system 100.

The microfluidic channel 106 extends from an inlet 108 to a pair of outlets 110A, 110B, wherein the inlet 108 and the outlet 110A, 110B may for example be arranged on the top surface of the substrate 104. The inlet 108 and the outlets 110A, 110B are configured to be connected to respective reservoirs (not shown) and/or pumps (not shown), e.g. to supply a carrier fluid containing the sample objects 102A, 102B to the inlet 108 and to withdraw the carrier fluid from the outlets 110A, 110B. The microfluidic channel 106 splits into a pair of sorting channels 106A, 106B at a sorting junction 112, wherein a first sorting channel 106A is in fluid communication with a first outlet 110A and a second sorting channel 106B is in fluid communication with a second outlet 110B.

The microfluidic channel 106, coming from the inlet 108, extends through a first/upstream detection zone 114, further through a manipulation zone 116 downstream of the upstream detection zone 114 and, as the sorting channel 106A, through a second/downstream detection zone 118 downstream of the manipulation zone 116 to the outlet 110A. In the upstream detection zone 114, a first/upstream electrode assembly 120 is arranged for performing electrical measurements on sample objects 102A, 102B within the microfluidic channel 106 in the upstream detection zone 114. The microfluidic system 100 further comprises means for manipulating sample objects in the manipulation zone 116, namely a manipulation electrode assembly 122 for generating an electric field within the microfluidic channel 106 in the manipulation zone 116 for selectively manipulating trajectories of the sample objects 102A, 102B by dielectrophoresis, i.e. motion in a spatially non-uniform electric field. In the downstream detection zone 118, a second/downstream electrode assembly 124 is arranged for performing electrical measurements on sample objects 102A, 102B within the sorting channel 106A in the downstream detection zone 114.

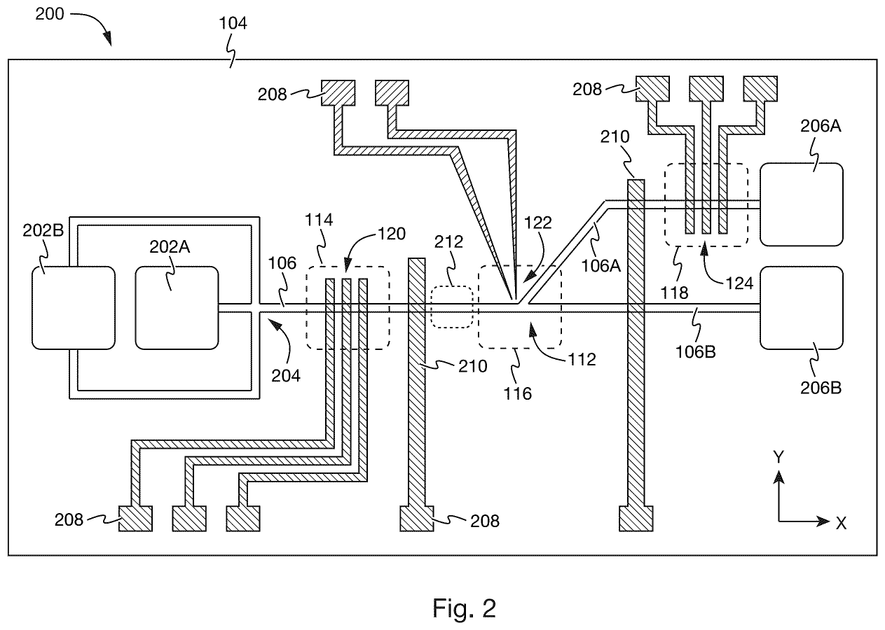

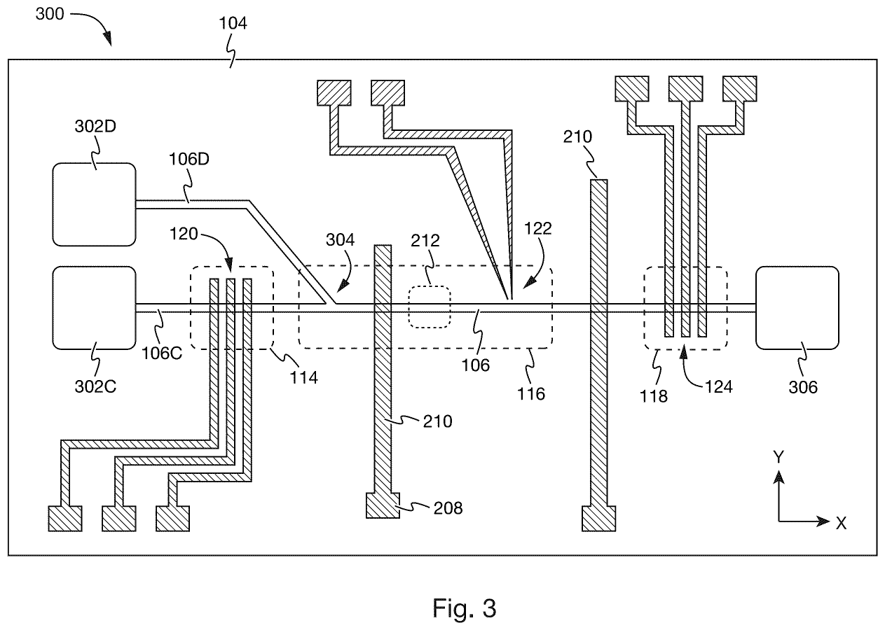

In the example of FIG. 1, the upstream electrode assembly 120 and the downstream electrode assembly 124 each comprise a pair of electrodes arranged on opposite sides of the microfluidic channel 106, wherein each of the electrodes may be in electrical contact with an inner volume of the microfluidic channel 106. Similarly, the manipulation electrode assembly 122 also comprises a pair of electrodes arranged on opposite sides of the microfluidic channel 106 in the manipulation zone 116, wherein each of the electrodes may be electrically isolated from the inner volume of the microfluidic channel 106. In other embodiments, the pair of electrodes of the manipulation electrode assembly 122 may also be arranged on the same side of the microfluidic channel 106, e.g. as in the examples of FIGS. 2 and 3.

FIG. 2 depicts a schematic illustration (not to scale) of a microfluidic system 200 for manipulating sample objects (not shown) according to another exemplary embodiment of the invention in top view. The microfluidic system 200 may e.g. be used for sorting sample objects such as microdroplets. The microfluidic system 200 may for example be operated using a method of operating a microfluidic system according to any one of the embodiments described herein, for example the method 500 of FIG. 5, and/or using a control system for operating a microfluidic system according to any one of the embodiments described herein, for example the control system 400 of FIG. 4.

The microfluidic system 200 is similar to the microfluidic system 100 of FIG. 1, wherein corresponding elements are denoted using the same reference signs as in FIG. 1. In particular, the microfluidic system 200 also comprises a microfluidic channel 106 that extends from a first/upstream detection zone 114 through a manipulation zone 116 to a second/downstream detection zone 118. In the manipulation zone 116, the microfluidic channel 106 splits at a sorting junction 112 into a pair of sorting channels, namely a target channel 106A and a waste channel 106B, with the downstream detection zone 118 being arranged along the target channel 106A. The microfluidic channel 106 may for example have a cross-sectional area between 10 μm² and 10 mm², in some examples between 100 μm² and 0.1 mm². The microfluidic channel may for example have a width between 10 μm and 0.5 mm and a height between 10 μm and 0.5 mm. A length of the microfluidic channel 106 may for example be between 1 mm and 100 mm.

The microfluidic system 200 further comprises a first reservoir 202A and a second reservoir 202B, each of which is configured to store a liquid. The reservoirs 202A, 202B are in fluid communication with a droplet generator 204, at which a pair of channels coming from the second reservoir 202B intersects under an angle with a channel coming from the first reservoir 202A. The droplet generator 204 may for example be configured to generate microdroplets (not shown) of a liquid (e.g. an aqueous solution) stored in the first reservoir 202A forming a dispersed phase in a continuous phase formed by a liquid (e.g. an oil) stored in the second reservoir 202B. In some embodiments, the liquid stored in the first reservoir may contain objects such as cells or one or more substances such as one or more reactants for a chemical reaction that are to be encapsulated in the microdroplets.

The microfluidic system 200 also comprises a target reservoir 206A and a waste reservoir 206B. The target reservoir 206A is in fluid communication with the target channel 106A and may for example be used to collect sample objects of a first type (target objects), e.g. microdroplets that contain a single cell. The waste reservoir 206 is in fluid communication with the waste channel 106B and may for example be used to collect sample objects/microdroplets that are not of the first type (waste objects), e.g. microdroplets of a second type such as microdroplets that do not contain a cell and/or microdroplets of a third type such as microdroplets that contain more than one cell.

A first/upstream electrode assembly 120 is arranged in the upstream detection zone 114. The upstream electrode assembly 120 comprises three electrodes that are arranged adjacent to each other and are each in electrical contact with the inner volume of the microfluidic channel 106 in the upstream manipulation zone 114. Similarly, a second/downstream electrode assembly 124 with three adjacent electrodes is arranged in the downstream detection zone 118. Each of the electrodes of the upstream and downstream electrode assemblies 120, 124 is electrically connected with a respective contact pad 208 for contacting the electrode. A distance between adjacent electrodes in the upstream and downstream electrode assemblies 120, 124 may for example be between 1 μm and 500 μm, in some examples between 5 μm and 200 μm, in one example between 10 μm and 100 μm. The distance between adjacent electrodes may for example be chosen based on a size of the sample objects, e.g. such that the distance is equal to or smaller than the size of the sample objects.

In some embodiments, the electrodes of the upstream and downstream electrode assemblies 120, 124 are formed on or in a top surface of an electrode substrate 104A (cf. FIG. 4), e.g. by depositing a conductive material such as titanium or platinum or gold thereon. The microfluidic structures of the microfluidic system 200 such as the microfluidic channel 106 may be formed in or on a bottom surface of a channel substrate 104B (cf. FIG. 4), e.g. by forming corresponding cutouts or recesses therein. The channel substrate 104B may be bonded to the electrode substrate 104A to form the substrate 104 of the microfluidic system 200, e.g. such that the top surface of the electrode substrate 104A is in contact with the bottom surface of the channel substrate (104B). After bonding, portions of the electrodes may be exposed to the inner volume of the microfluidic channel 106. A surface area of each of the electrodes that is exposed to the inner volume of the microfluidic channel 106 may for example be between 10 μm² and 1 mm², in some examples between 100 μm² and 0.1 mm². Preferably, the exposed surface areas of the electrodes of a given electrode assembly are the same or substantially the same.

The microfluidic system 200 further comprises a pair of ground electrodes 210, which are also in electrical contact with the inner volume of the microfluidic channel 106 and may for example be formed in the same way as the electrodes of the upstream and downstream electrode assemblies 120, 124. The ground electrodes 210 may for example be used for providing a reference voltage, e.g. a ground potential, to the microfluidic system 200.

In the manipulation zone 116, a manipulation electrode assembly 122 is arranged, which comprises a pair of tip-shaped electrodes that are arranged in close vicinity to a portion of the microfluidic channel 106 at or slightly upstream of the sorting junction 112. In other embodiments, the manipulation electrode assembly 122 may additionally or alternatively comprise electrodes with a different, shape, e.g. a pair of planar or flat-shaped electrodes as in the example of FIG. 1 or a combination of planar and flat-shaped electrodes. The manipulation assembly 122 may be configured to generate a spatially non-uniform electric field at the sorting junction 112, e.g. to selectively deflect microdroplets of the first type into the target channel 106A by dielectrophoresis. The manipulation electrode assembly 122 may for example be arranged in and/or on the channel substrate 104B. In other embodiments, the microfluidic system 200 may comprise other means for manipulating the sample objects in the manipulation zone either in addition to or instead of the manipulation electrode assembly 122, for example one or more inductive elements for generating a magnetic field, an acoustic resonator for generating a standing acoustic wave, a lateral channel and a valve for generating a transverse deflecting flow, and/or an illumination window for generating an optical potential, which may e.g. be similar to or may be the detection window 212 described below.

The microfluidic system 200 further comprises a detection window 212 that is arranged upstream of or in the manipulation zone 116. The detection window 212 is configured for optical imaging, in particular microscopic imaging, of sample objects in the microfluidic channel 106 and may for example comprise or consist of an optically transparent material such as a transparent thermoplastic like PMMA or a glass such as fused silica or BK7 glass. In some embodiments, the detection window 212 may be formed by or arranged in and/or on the electrode substrate 104A, the channel substrate 104B or a cover plate (not shown) of the substrate 104. The detection window 212 may for example be used for detecting a fluorescence signal emitted by sample objects and/or for spatially resolved microscopic imaging of the sample objects. The fluorescence signal and/or the microscopic image may for example be used for classifying the sample objects, e.g. to decide whether or not to deflect a given sample object into the target channel 106A using the manipulation electrode assembly 122.

FIG. 3 depicts a schematic illustration (not to scale) of a microfluidic system 300 for manipulating sample objects (not shown) according to another exemplary embodiment of the invention in top view. The microfluidic system 300 is similar to the microfluidic system 200 of FIG. 2, wherein corresponding elements are denoted using the same reference signs as in FIG. 2 and description thereof is omitted for the sake of brevity.

The microfluidic system 300 may for example be used for merging sample objects to form composite sample objects. The microfluidic system 300 comprises a first reservoir 302C and a second reservoir 302D, each of which is configured to store a liquid containing sample objects. For example, the first reservoir 302C may be used for providing sample objects of a first type and the second reservoir 302D may be used for providing sample objects of a second type. In some embodiments, the microfluidic system 300 may also comprise a respective droplet generator instead of or in addition to one or both of the reservoirs 302C, 302D, e.g. to generate microdroplets of a first type and microdroplets of a second type.

The microfluidic channel 106 extends from the first reservoir 302C and the second reservoir 302D to a target reservoir 306, which may for example be used for collecting the composite sample objects. The microfluidic system 300 comprises a merging junction 304, at which a portion or sub-channel 106C of the microfluidic channel 106 that is connected to the first reservoir 302C merges with another portion or sub-channel 106D of the microfluidic channel 106 that is connected to the second reservoir 302D. The upstream detection zone 114 with the upstream electrode assembly 120 is arranged along the sub-channel 106C of the microfluidic channel 106, i.e. such that the merging junction 304 is arranged between the upstream detection zone 114 and the manipulation electrode assembly 122.

The manipulation electrode assembly 122 may for example be configured to generate an electric field in the manipulation zone 116 for merging a sample object of the first type with a sample object of the second type to form a composite sample object. The electric field may for example generate forces on the sample object of the first type and/or on the sample object of the second type such that the respective sample objects collide or come in contact with each other and merge or fuse into the composite sample objects, e.g. as a result of electro-coalescence. The intersection of the sub-channels 106C, 106D at the merging junction 304 may for example be designed such that incoming sample objects from the sub-channels 106C, 106D may come in close vicinity to each other at the merging junction 304 and flow together along the microfluidic channel 106 to the manipulation electrode assembly 122, by which the sample objects are then merged into the composite sample object. In other embodiments, the intersection of the sub-channels 106C, 106D at the merging junction 304 may be designed such that incoming sample objects from the channels 106C, 106D may collide at the merging junction 304 and already merge into composite sample objects at the merging junction 304.

In the example of FIG. 3, the downstream detection zone 118 with the downstream electrode assembly 124 is arranged along the microfluidic channel 106 between the manipulation electrode assembly 122 and the target reservoir 306. In other examples, the second/downstream detection zone 118 may be arranged differently, for example at the merging junction 304, between the merging junction 304 and the manipulation electrode assembly 122, or along the sub-channel 106D of the microfluidic channel 106 between the second reservoir 302D and the merging junction 304.

FIG. 4 depicts a schematic illustration (not to scale) of a control system 400 for operating a microfluidic system according to an exemplary embodiment of the invention in side view. The control system 400 may be used for operating a microfluidic system comprising a manipulation zone 116 for manipulating sample objects (not shown) flowing through the manipulation zone 116, a first/upstream detection zone 114 arranged upstream of or in the manipulation zone 116 and a second/downstream detection zone 118 arranged upstream of, in or downstream of the manipulation zone 116. The control system 400 may in particular be used for operating a microfluidic system according to any one of the embodiments described herein, for example any one of the microfluidic systems 100, 200, and 300 described above. In the example of FIG. 4, the control system 400 is used for operating a microfluidic system 200′, which is similar to the microfluidic system 200 and used as a non-limiting example for illustration purposes in the following. The control system 400 may be used for executing a method of operating a microfluidic system according to any one of the embodiments described herein, for example the method 500 of FIG. 5. In some embodiments, the control system 400, in particular the controller 408 of the control system 400, may be configured to execute some or all of the steps of a method of operating a microfluidic system according to any one of the embodiments described herein such as the method 500 of FIG. 5.

The control system 400 comprises a mount 402 that is configured to receive the microfluidic system 200′. The microfluidic system 200′ is similar to the microfluidic system 200 of FIG. 2, but—similar to the microfluidic system 100 of FIG. 1—comprises an inlet 108 instead of the reservoirs 202A, 202B and the droplet generator 204 and comprises a pair of outlets 110A, 110B instead of the reservoirs 206A, 206B. The mount 402 is configured to hold the microfluidic system 200′ in place. The mount 402 may also be configured to provide electrical and/or fluid connections (not shown) to the microfluidic system 200′, for example to contact pads (not shown) of the upstream electrode assembly 120, the manipulation electrode assembly 122, and/or the downstream electrode assembly 124, and/or to the inlet 108 and/or the outlets 110A, 110B of the microfluidic channel 106. The mount 402 may also be configured to move and/or tilt the microfluidic system 200′, e.g. to align the microfluidic system 200′ relative to the imaging system of the sorting unit 410 described below.

The control system 400 further comprises a measurement unit 404 that is configured to perform optical, acoustic and/or electrical measurements in the upstream detection zone 114 and in the downstream detection zone 118 for detecting sample objects (not shown) in the respective detection zone 114, 118. In the example of FIG. 4, the measurement unit 404 is configured to perform differential electrical measurements, in particular differential impedance measurements, between pairs of electrodes in each of the detection zones 114, 118, for example between a pair comprising a left electrode and a center/reference electrode and a pair comprising a right electrode and a center/reference electrode of the respective electrode assembly 120, 124. For this, the measurement unit 404 may for example comprise a voltage source (not shown) for applying voltages to each of the pairs and an ammeter (not shown) for measuring currents through each of the pairs. The ammeter may for example comprise a transimpedance amplifier, in particular a differential transimpedance amplifier.

Additionally or alternatively, the measurement unit 404 may also be configured to perform optical measurements in the upstream detection zone 114 and/or in the downstream detection zone 118 for detecting sample objects. For this, the measurement unit 404 may comprise an imaging system similar to the imaging system of the sorting unit 410 described below and/or may share an imaging system with the sorting unit 410. In some embodiments, the upstream detection zone 114 and/or the downstream detection zone 118 may for example each be a region of interest in an image (in one example in the same image) of the microfluidic system 200′ or a part thereof, for example an image of the manipulation zone 116 and/or through the detection window 212.

The control system 400 also comprises a manipulation unit 406 configured to apply a force on sample objects in the manipulation zone 116 for manipulating the sample objects. The manipulation unit 406 may in particular be configured to manipulate the sample objects as described below for step 504 of the method 500, e.g. by executing step 504. The manipulation unit 406 may for example comprise a voltage source (not shown) that is configured to apply a voltage, in particular a voltage pulse or a pulse sequence, to the manipulation electrode assembly 122, e.g. to manipulate trajectories of the sample objects by dielectrophoresis. The manipulation of the sample objects in the manipulation zone 116 is characterized by a plurality of manipulation parameters x₁, x₂, . . . that affect how the sample objects are manipulated. The force exerted on the sample objects may for example depend on the values of these manipulation parameters. The force may e.g. depend on parameters of the applied voltage such as an amplitude A_{V}, a frequency f_{V}, a pulse duration P_{V}, and a pulse delay L_{V}. The pulse delay may for example quantify the delay between a reference point in time and the start of the pulse. The reference point in time may for example be the point in time at which a sample object is detected in the upper detection zone 114, in the manipulation zone 116 or in a detection zone associated with the detection window 212. In other embodiments, the reference point in time may for example be the point in time at which the sample object is classified, at which a decision is made as to whether or not to manipulate the sample object or at which a sorting control signal is received by the manipulation unit 406.

The control system 400 further comprises a controller 408, which may be implemented in hardware, software, or a combination thereof. The controller 408 may for example comprise one or more processing devices such as a central processing unit (CPU), a graphics processing unit (GPU), an application specific integrated circuit (ASIC), and/or a field-programmable gate array (FPGA), and one or more storage media, e.g. a non-volatile memory and/or a volatile memory, containing instructions for execution by the processing device to provide the functionality described herein. In some embodiments, the measurement unit 404, the manipulation unit 406, or parts thereof may be integrated into the controller 408, i.e. the controller 408 may be configured to provide the respective functionality or at least a part thereof.

The controller 408 is configured to determine a first count N₁ characterizing a number of sample objects flowing through the upstream detection zone 114 (i.e. (i.e. an input count Nᵢₙ characterizing a number of non-manipulated sample objects prior to manipulation in the manipulation zone 116) and a second count N₂ characterizing a number of sample objects flowing through the downstream detection zone 118 (i.e. an output count Nₒᵤₜ characterizing a number of manipulated sample objects after manipulation in the manipulation zone 116) based on the optical, acoustic and/or electrical measurements performed by the measurement unit 404. The controller 408 is further configured to adjust one or more of the manipulation parameters x₁, x₂, . . . associated with the force applied by the manipulation unit 406 on the sample objects in the manipulation zone 116 based on the first count N₁ and the second count N₂. The controller 408 may in particular be configured to determine the first count and the second count and to adjust the one or more manipulation parameters by executing the respective parts of the method 500 described below, e.g. steps 502, 506, and/or 508. The controller 408 may further be configured to execute some or all of the other parts of the method 500, for example to manipulate sample objects in the manipulation zone 116 as in step 504. For this, the controller 408 may be configured to control some or all of the other components of the control system 400, in particular the measurement unit 404, the manipulation unit 406, the sorting unit 410, and/or the microfluidics unit 418.

The control system 400 further comprises a sorting unit 410 for sorting the sample objects at the sorting junction 112. The sorting unit 410 comprises an imaging system for performing optical measurements on sample objects in the microfluidic channel 106 through the detection window 212. The imaging system comprises a light source 412 such as a laser for illuminating the sample objects through the detection window 212 and an objective, in particular a high-NA objective 414 for imaging the sample objects through the detection window 212 onto a photodetector 416 such as a photodiode or CMOS chip configured to measure an intensity of light. The sorting unit 410 is configured to classify individual sample objects based on the optical measurements, e.g. based on one or more thresholds for the intensity of light emitted, transmitted, or reflected by the respective sample object, a size of the respective sample object, and/or a shape of the respective sample object. This functionality of the sorting unit 410 may for example be provided by the controller 408 or by a sub-controller (not shown) of the sorting unit 410. The sorting unit 410 is further configured to guide the sample objects in the manipulation zone 116 into either the target channel 106A or the waste channel 106B based on the classification by controlling the manipulation unit 406 accordingly, e.g. by generating a corresponding sorting control signal for the manipulation unit 406.

The control system 400 also comprises a microfluidics unit 418, which is configured to generate a fluid flow along the microfluidic channel 106. For this, the microfluidics unit 418 may for example comprise one or more pumps (not shown), one or more valves (not shown), and/or one or more reservoirs (not shown) to supply fluid to the inlet 108 and/or to withdraw fluid from one or both of the outlets 110A, 110B. Operation of the microfluidics unit 418, e.g. of the pumps and/or the valves, may be controlled by a plurality of control parameters y₁, y₂, . . . , which may for example determine one or more flow rates of fluids, for example an input flow rate of a dispersed phase, an input flow rate of a continuous phase, a withdrawal rate at the outlet 110A, and/or a withdrawal rate at the outlet 110B. The controller 408 may be configured to adjust the control parameters y₁, y₂, . . . . In some embodiments, some or all of the control parameter y₁, y₂, . . . may also be manipulation parameters that are adjusted based on the input count and the output count.

FIG. 5 shows a flow chart of a method 500 of operating a microfluidic system according to an exemplary embodiment of the invention. The method 500 may for example be used for operating the microfluidic system 200 of FIG. 2, which is used as an example for illustration purposes in the following. This is, however, not intended to be limiting in any way and the method 500 may also be used for operating any other microfluidic system comprising a manipulation zone for manipulating sample objects flowing through the manipulation zone, an upstream detection zone arranged upstream of or in the manipulation zone and a downstream detection zone arranged downstream of the upstream detection zone, for example the microfluidic system 100 of FIG. 1, the microfluidic system 300 of FIG. 3, the microfluidic system 200′ of FIG. 4 or any other microfluidic system according to one of the embodiments described herein. The method 500 may for example be executed using a control system for operating a microfluidic system according to any one of the embodiments disclosed herein such as the control system 400 of FIG. 4, which is also used as a non-limiting example for illustration purposes in the following. The method 500 is not limited to the order of execution indicated by the flow chart of FIG. 5. As far as technically feasible, the method 500 may be executed in an arbitrary order and parts thereof may be executed simultaneously at least in part.

The method 500 may for example be used for sorting sample objects such as microfluidic droplets, e.g. to separate microdroplets of a first type from microdroplets of a second type. This is used as a non-limiting example for illustration purposes in the following. Additionally or alternatively, the method 500 may be used to manipulate other types of sample objects, e.g. microbubbles, solid microparticles or cells, and/or to perform other types of manipulations on sample objects, e.g. to merge two or more sample objects into a composite sample objects or to manipulate trajectories and/or properties of sample objects in other ways.

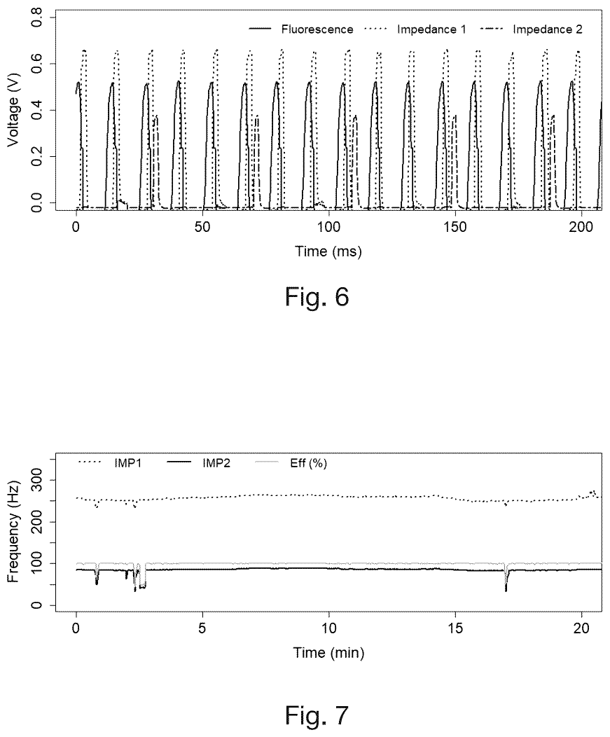

The method 500 comprises, in step 502, determining a first count N₁ that characterizes a number of microdroplets flowing through the first/upstream detection zone 114 (i.e. an input count Nᵢₙ that characterizes a number of non-sorted microdroplets prior to the sorting). For example, an electrical measurement such as an impedance measurement may be performed in the upstream detection zone 114 using the upstream electrode assembly 120. An exemplary time trace of a measurement signal recorded by performing a differential impedance measurement comparing a current through the left electrode of the upstream electrode assembly 120 to a current through the right electrode when applying a voltage at these electrodes relative to the center/reference electrode is depicted as a dotted line (“Impedance 1”) in FIG. 6. Microdroplets flow through the upstream detection zone 114 at a constant input rate f_{D} such that the measurement signal exhibits a plurality of regularly spaced peaks, each of which is a signature associated with the passage of a single microdroplet through the upstream detection zone 114 (e.g. because the microdroplets exhibit a different electrical conductivity than a surrounding medium such as the carrier fluid/continuous phase in which the microdroplets are transported). By comparing the measurement signal to a pre-defined threshold, individual microdroplets may be detected. Based on this, the input count Nᵢₙ may be determined, wherein the input count may e.g. characterize the input rate of the microdroplets, which may for example be between 1 Hz and 1 kHz. Besides detecting microdroplets, the measurement signal may also allow for determining properties of the microdroplets such as their size or velocity, e.g. based on a duration/width and/or an amplitude/height of the corresponding peak.

The method 500 further comprises, in step 504, manipulating microdroplets in the manipulation zone 116, e.g. sorting the microdroplets by selectively deflecting the microdroplets into either the target channel 106A or the waste channel 106B. This may for example be done by performing optical measurements on the microdroplets through the detection window 212 using the imaging system of the sorting unit 410, e.g. by recording a fluorescence intensity emitted by the microdroplets. An exemplary time trace of a fluorescence intensity recorded by the sorting unit 410 is shown as a solid line (“Fluorescence”) in FIG. 6. Similar to the measurement signal recorded with the upstream electrode assembly, the measured fluorescence intensity exhibits a plurality of regularly spaced peaks, each of which is a signature associated with the passage of a single microdroplet past the detection window 212.

In some embodiments, this optical measurement may also be used to determine the input count in step 502 instead of or in addition to the electrical measurement described above. In other words, the upstream detection zone 114 may be located at the detection window 212 in some embodiments and may e.g. be a part of the manipulation zone 116. In some examples, one or both of the upstream and downstream detection zones 114, 118 may each be a region of interest in an image (in one example in the same image) of the microfluidic system 200 or a part thereof, for example an image of the manipulation zone 116 and/or through the detection window 212.

Based on the measured fluorescence intensity, the microdroplets may be classified, for example by determining whether a given microdroplet is a microdroplet of the first type or a microdroplet of the second type, e.g. using the sorting unit 410. For example, microdroplets of the first type may exhibit stronger fluorescence than microdroplets of the second type (not shown). This may allow for classifying the microdroplets using one or more thresholds or ranges for the fluorescence intensity, e.g. by assigning a detection event associated with a fluorescence intensity above a classification threshold to a microdroplet of the first type and a detection event associated with a fluorescence intensity below the classification threshold, but above a detection threshold to a microdroplet of the second type.

Based on the classification, the sorting unit 410 may generate a sorting control signal for the manipulation unit 406 to either deflect the respective microdroplet into the target channel 106A by applying a voltage pulse or pulse sequence to the manipulation electrode assembly 122 or to let the respective microdroplet pass through the sorting junction 112 unhindered into the waste channel 106B.

In step 506, a second count N₂ is determined that characterizes a number of microdroplets flowing through the downstream detection zone 118 arranged along the target channel 106A (i.e. an output count Nₒᵤₜ that characterizes a number of sorted microdroplets after the sorting). The second/output count may be determined in substantially the same way as the first/input count, e.g. by performing an electrical measurement such as an impedance measurement in the downstream detection zone 118 using the downstream electrode assembly 124.

The dash-dotted line (“Impedance 2”) in FIG. 6 depicts an exemplary time trace of a measurement signal recorded by performing a differential impedance measurement comparing a current through the left electrode of the downstream electrode assembly 124 to a current through the right electrode when applying a voltage at these electrodes relative to the center/reference electrode.

The output count Nₒᵤₜ may be used for monitoring the manipulation performed in step 504, e.g. to monitor whether a microdroplet that should have been deflected into the target channel 106A according to the control signal does indeed end up in the target channel 106A. This may for example comprise determining an efficiency metric η for the manipulation/sorting based on the input count Nᵢₙ and/or the output count Nₒᵤₜ. The efficiency metric may e.g. characterize a ratio between the actual output count Nₒᵤₜ and an expected output count, which may for example be determined based on the fluorescence signal and/or the sorting control signal. An efficiency metric below one (η<1) may for example indicate that the deflection of the microdroplets is not working properly.

The method 500 further comprises, in step 508, adjusting one or more manipulation parameters for manipulating the sample objects in the manipulation zone 116 based on the input count Nᵢₙ and the output count Nₒᵤₜ. This may for example comprise adjusting one or more of the manipulation parameters x₁, x₂, . . . associated with the manipulation unit 406, e.g. one or more of the amplitude A_{V}, the frequency f_{V}, the pulse duration P_{V}, and the pulse delay L_{V} of the voltage applied to the manipulation electrode assembly 122. Additionally or alternatively, this may also comprise adjusting one or more of the control parameters y₁, y₂, . . . associated with the microfluidics unit 418, for example one or more of an input flow rate Q_{D} of the dispersed phase at the droplet generator 204, an input flow rate Q_{C} of the continuous phase at the droplet generator 204, and/or the withdrawal rate Q_{w} of the continuous phase and the dispersed phase through the waste channel 106B. The manipulation parameters may for example be adjusted for optimizing the efficiency metric η in step 508A (training mode), e.g. as part of an initialization procedure for the control system 400, and/or for implementing a feedback control for the manipulation during normal operation (screening mode) of the control system 400 in step 508B.

Feedback control of one or more of the manipulation parameters in step 508B may for example be implemented by monitoring the input count Nᵢₙ and the output count Nₒᵤₜ continuously during operation of the microfluidic system 200, e.g. by repeated execution of steps 502 to 506. The current output count Nₒᵤₜ and/or the current efficiency q may for example be compared to a set point or set range, e.g. a pre-defined range around the respective initial value, which may for example have been obtained by optimization in step 508A as described below. If a deviation occurs, the manipulation parameters that are to be controlled may be adjusted to reduce the deviation and/or until the set point or set range is reached. Feedback control may for example be performed for one or more of the amplitude A_{V}, a frequency f_{V}, a pulse duration P_{V}, and a pulse delay L_{V} of the voltage, e.g. at least for the pulse delay, at least for the pulse delay and the pulse duration, or at least for the pulse delay, the pulse duration, and the amplitude.

Feedback control of a given manipulation parameter may be implemented using any control loop known in the art, for example a proportional control loop, a proportional-integral (PI) control loop, and/or a proportional-integral-derivative (PID) control loop. In some embodiments, different gains and/or time constants may be employed for different manipulation parameters. For example, feedback control may be implemented for the pulse duration P_{V} and the pulse delay L_{V}, but using a higher gain and/or smaller time constant for the pulse delay than for the pulse duration, e.g. such that the feedback loop at first attempts to return to the set point by predominantly adjusting the pulse delay rather than the pulse duration.

FIG. 7 depicts exemplary time traces of an input count/input rate (“IMP1”, dotted line), an output count/output rate (“IMP2”, solid black line), and an efficiency metric (“Eff (%)”, solid grey line) during operation of a microfluidic system such as the microfluidic system 200 with continuous feedback control in step 508B. In spite of variations in conditions over time, as e.g. exemplified by slow drifts in the input rate of the microdroplets, a high and substantially constant sorting efficiency can be achieved. Intermittent failures such as pulsations or failure of a pump or clogging to the microfluidic channel 106 may also be detected by monitoring the input count, the output count, and/or the sorting efficiency, e.g. by looking for sudden drops in the sorting efficiency. If a failure occurs, operation of the microfluidic system may e.g. be paused temporarily or terminated indefinitely.

Optimizing the efficiency metric in step 508A may for example comprise scanning one or more of the manipulation parameters, e.g. one or more of the parameter x₁, x₂, . . . and/or one or more of the parameters y₁, y₂, . . . to obtain an optimized set of manipulation parameters. The optimized set of manipulation parameters may e.g. be associated with an optimum of the efficiency metric and/or with an efficiency metric above or below a pre-defined threshold, e.g. a minimum manipulation efficiency that is to be achieved. The optimized set of manipulation parameters may subsequently be used during operation of the microfluidic system 200, e.g. in step 508B.

For example, one or more of the amplitude A_{V}, the frequency f_{V}, the pulse duration P_{V}, and the pulse delay L_{V} of the voltage applied to the manipulation electrode assembly 122 may be scanned to determine an optimized set of these parameters (A_{V}, f_{V}, P_{V}, L_{V}). This optimization may for example be performed for a particular input rate f_{D} of the microdroplets, e.g. by keeping the input flow rate Q_{D} of the dispersed phase, the input flow rate Q_{C} of the continuous phase, and the withdrawal rate Q_{w} through the waste channel 106B constant. In some embodiments, the optimization may be repeated at one or more other input rates f_{D}, e.g. to obtain sets of optimized manipulation parameters (A_{V}, f_{V}, P_{V}, L_{V}) for a plurality of input rates, to optimize the efficiency metric further to obtain a set of optimized manipulation parameters (A_{V}, f_{V}, P_{V}, L_{V}, f_{D}) or (A_{V}, f_{V}, P_{V}, L_{V}, Q_{D}, Q_{C}, Q_{w}), or to determine a maximum flow rate at which a certain optimization criterion ηₑ (e.g. a minimum manipulation efficiency) can be met.

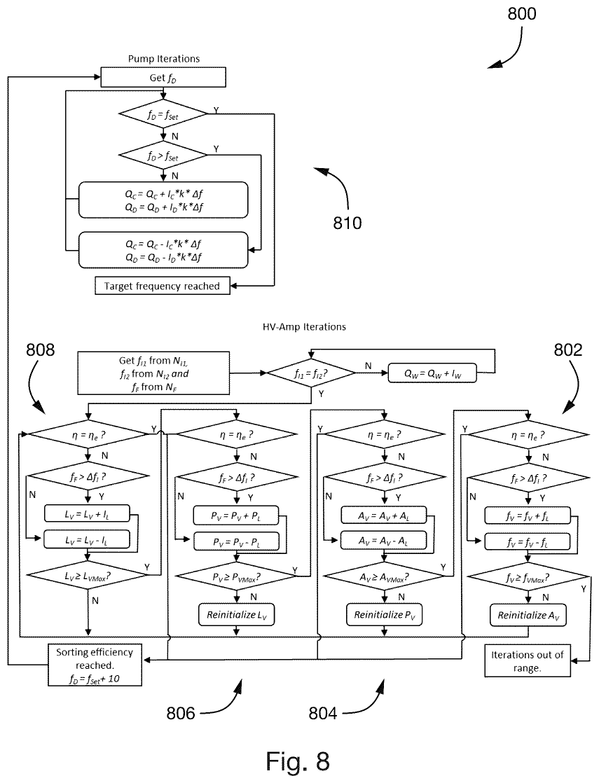

FIG. 8 shows a flow chart of a method 800 of optimizing an efficiency metric η in accordance with an exemplary embodiment of the invention, which may for example be executed in step 508A. The method 800 may be used to obtain sets of optimized manipulation parameters (A_{V}, f_{V}, P_{V}, L_{V}) at a given input rate f_{D}, for which the efficiency metric η is at least a minimum manipulation efficiency. The method 800 comprises four nested iteration loops, namely four nested for-loops 802-810, each of which is used to scan a respective manipulation parameter from a lower bound to an upper bound or vice-versa to perform a multi-dimensional raster scan of the manipulation parameters.

A first level for-loop 802 iteratively increases or decreases the frequency f_{V} of the applied voltage by a pre-defined iteration step f_{L} and during each iteration executes a second level for-loop 804 (“reinitialize A_{V}”). The second level for-loop 804 iteratively increases or decreases the amplitude A_{V} of the applied voltage by a pre-defined iteration step A_{L} (at constant f_{V}) and during each iteration executes a third level for-loop 806 (“reinitialize P_{V}”). The third level for-loop 806 iteratively increases or decreases the pulse duration P_{V} of the applied voltage by a pre-defined iteration step P_{L} (at constant f_{V} and A_{V}) and during each iteration executes a fourth level for-loop 808 (“reinitialize L_{V}”). The fourth level for-loop 808 iteratively increases or decreases the pulse delay L_{V} of the applied voltage by a pre-defined iteration step l_{L} (at constant f_{V}, A_{V} and P_{V}). During each iteration of one of the for-loops 802-810, the efficiency metric η is determined and compared to a minimum sorting efficiency ηₑ that is to be achieved (“η=ηₑ?”). If the minimum sorting efficiency ηₑ is reached, the scan terminates yielding a set of optimized manipulation parameters (A_{V}, f_{V}, P_{V}, L_{V}) for the respective input rate f_{D}. Otherwise, the scan continues.

The optimization may be repeated for one or more other input flow rates, e.g. by implementing a corresponding zero-th level for-loop. To achieve a given input rate f_{D}, a feedback loop 810 may be executed, which may for example adapt the input flow rate Q_{D} of the dispersed phase and the input flow rate Q_{C} of the continuous phase until the desired input rate f_{D} is reached.

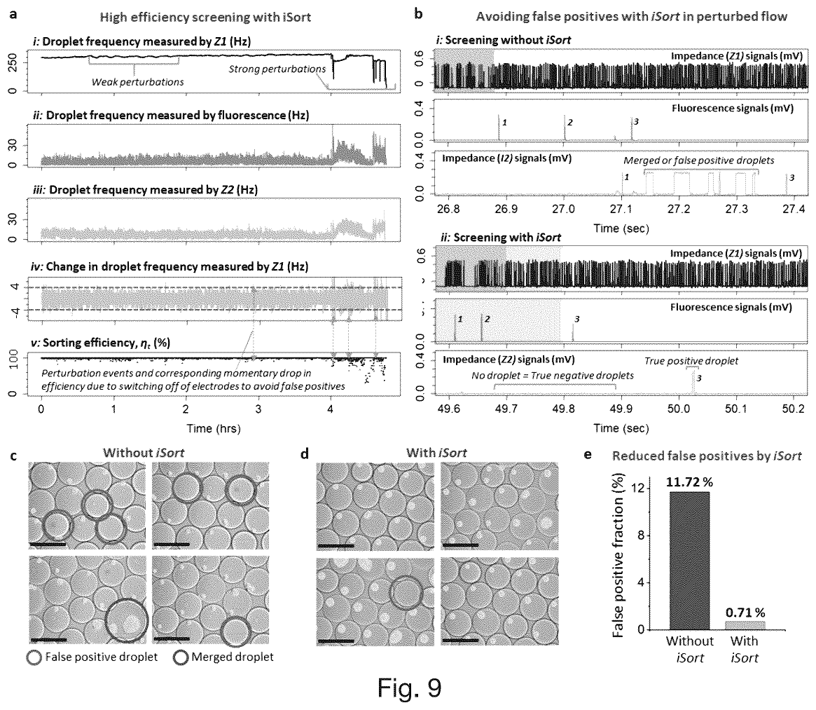

FIGS. 9a to 9e show the results of screening experiments in which droplets containing fluorescent beads were sorted using a method and a microfluidic system according to exemplary embodiments of the invention (iSort algorithm) along with a back-to-back comparison of this method (iSort algorithm) to a conventional screening method (without feedback loops), see FIGS. 9b and 9e, which directly compare the two approaches, and FIGS. 9c and 9d showing outcomes of the conventional method and the iSort algorithm, respectively. The results demonstrate the capacity to monitor and ensure efficient and long-term sorting. For this, green fluorescent beads having a diameter of about 8 μm were encapsulated into droplets generated by a droplet generator in the microfluidic system at an average occupancy of about 0.05 beads per droplet. A sorting operation for droplets showing green fluorescence signals of the beads was performed using sets of optimized manipulation parameters from a configuration file determined beforehand in scanning/training mode.

FIG. 9a shows the frequency profile of the droplets as measured by different sensors during the screening and the corresponding sorting efficiency, demonstrating the iSort working principle and resulting data. Panel i depicts the frequency of all the droplets measured by primary impedance signals (Z1, as an example of a first/input count). Panel ii depicts the frequency of positive droplets (droplets containing green fluorescent beads) measured by fluorescence signals. Panel iii depicts the frequency of droplets in the collection channel measured by secondary impedance signals (Z2, as an example of a second/output count). Panel iv depicts the change in frequency (i.e. the difference between the current frequency and the frequency measured one second ago) measured by Z1 with a threshold (dashed line) set at f 4 Hz. This threshold is used to decide if the perturbations require pausing of the sorting process (i.e. switching off the high-voltage pulse used for inducing a dielectrophoretic force for sorting the droplets) to avoid false positives and merged droplets (as an example for a determination whether a failure has occurred during operation of the microfluidic system). Panel v depicts the efficiency ηₜ of the droplet sorting during the complete screening process, wherein ηₜ=(Nₘ−N_{E})/N_{E} with Nₘ and N_{E} being the measured and expected number, respectively, of droplets every time the expected count N_{E} equals 100. The points where the efficiency is below 100% are mostly a result of the perturbations and pausing of sorting upon detection of these perturbations to avoid multiple false positive droplets at the cost of a few true positive droplets that cause the momentary efficiency drop. A few of such events and the corresponding perturbations are highlighted in the plot.

The screening continued for about 5 hours at an average throughput of 307 Hz and an average positive droplet frequency of 6.8 Hz as measured by fluorescence signals. The latter reciprocated with the droplet frequency measured in the collection channel by Z2, demonstrating high sorting efficiency. For the first four hours, the flow was relatively stable with a f 16 Hz variation in the throughput after which the perturbations were strong probably due to clogging by clustered beads. By the end of the screen, as the aqueous phase syringe (dispersed phase) got empty, the throughput gradually decreased and the screening was stopped. The varying throughput was compensated by the iSort algorithm by correcting the high-voltage parameters in real time in accordance with the configuration file (for e.g. if the throughput approached 320 Hz, the algorithm used the pulse amplitude A_{V}, the pulse duration D_{V} and the pulse delay L_{V} values suggested by the configuration file for 320 Hz). Overall, more than 4.2 million droplets were screened until the onset of terminal perturbations and 114,421 were counted in the collection channel, out of which, 113,798 were true positive droplets resulting in a purity (i.e. the fraction of true positive droplets in the collection) of 99.45% and an enrichment (i.e. the ratio of the fraction of true positives after and before sorting) of 45.8 fold. The pausing function which paused the sorting (i.e. stopped generating the high-voltage pulse) upon detection of perturbations for which the change in the droplet frequency measured by Z1 was 4 Hz (FIG. 9a, panel iv) contributed to the high purity. Pausing the sorting during strong perturbations ensures that the droplets did not encounter any dielectrophoretic force which could have resulted in sorting of multiple negative droplets along with the positive droplets or even merging of droplets due to small spacing in high-throughput screens. This way, pausing aims to prevent large numbers of false positive droplets at the expense of missing a few true positives.

FIG. 9b shows the results of a screening experiment demonstrating that iSort prevents false positive events to enable higher enrichment of true positives in comparison to a conventional screen (screening without iSort), conducted using the same microfluidic system under identical conditions. The plots show voltage signals collected from represented sensors where every peak corresponds to a droplet detection (the numbered peaks represent a given droplet detected by different sensors). The perturbations are highlighted in dark-grey shading and the time window where iSort paused the sorting upon detection of the perturbations is highlighted in light grey. Panel i shows the screening without iSort, demonstrating that perturbations cause multiple false positives and merged droplets. Panel ii shows the screening with iSort, demonstrating that iSort paused the sorting operation upon detection of perturbations to avoid false positives. FIG. 9c shows images of droplets collected from a screen in conventional mode (without iSort) showing multiple false positives and merged droplets (scale bar: 100 μm). FIG. 9d shows images of droplets collected from an iSort operated screen showing few false positives (scale bar: 100 μm). FIG. 9e shows a comparison of the fraction of false positive droplets in the collection from the screens operated with and without iSort. The fractions were calculated optically using microscope images of more than 500 droplets.

The conventional screen resulted in higher false positives and merged droplets (cf. FIGS. 9b-d). iSort successfully minimized events in which negative droplets enter the collection channel, in particular during strong perturbations. The droplets collected from these experimental screens were optically analyzed, confirming that iSort operated screens deliver higher enrichment with 16.5-fold less false positive events in comparison to conventional droplet screening setups. This shows that the present invention can successfully achieve high throughout droplet sorting with minimal false positives to ensure high enrichment factors of true positives in a fully automated way.

The embodiments of the present invention disclosed herein only constitute specific examples for illustration purposes. The present invention can be implemented in various ways and with many modifications without altering the underlying basic properties. Therefore, the present invention is only defined by the claims as stated below.

### LIST OF REFERENCE SIGNS

100—microfluidic system
102A—sample objects of a first type
102B—sample objects of a second type
104—substrate
104A—electrode substrate
104B—channel substrate
106—microfluidic channel
106A—target channel/sorting channel/downstream portion of the microfluidic channel 106
106B—waste channel/sorting channel/downstream portion of the microfluidic channel 106
106C—upstream portion/sub-channel of the microfluidic channel 106
106D—upstream portion/sub-channel of the microfluidic channel 106
108—inlet
110A—first outlet/target outlet
110B—second outlet/waste outlet
112—sorting junction
114—first/upstream detection zone
116—manipulation zone
118—second/downstream detection zone
120—first/upstream electrode assembly
122—manipulation electrode assembly
124—second/downstream electrode assembly
200, 200′—microfluidic system
202A—first reservoir (dispersed phase)
202B—second reservoir (continuous phase)
204—droplet generator
206A—target reservoir
206B—waste reservoir
208—contact pads
210—ground electrodes
212—detection window
300—microfluidic system
302C—first reservoir
302D—second reservoir
304—merging junction
306—target reservoir
400—control system
402—mount
404—measurement unit
406—manipulation unit
408—controller
410—sorting unit
412—light source
414—objective
416—photodetector
418—microfluidics unit
500—method of operating a microfluidic system
502—step of determining the first count N₁ (input count Nᵢₙ)
504—step of manipulating sample objects
506—step of determining the second count N₂ (output count Nₒᵤₜ)
508—step of adjusting manipulation parameters
508A—step of optimizing the efficiency metric η
508B—step of performing feedback control of manipulation parameters
800—method of optimizing the efficiency metric η
802—first level iteration loop for the frequency f_{V}
804—second level iteration loop for the amplitude A_{V}
806—third level iteration loop for the pulse duration P_{V}
808—fourth level iteration loop for the pulse delay L_{V}
810—feedback loop for the flow rate f_{D}
N₁ (Nᵢₙ)—first count (input count)
N₂ (Nₒᵤₜ)—second count (output count)
η—efficiency metric
ηₑ—optimization criterion
x₁, x₂, . . . —manipulation parameters
y₁, y₂, . . . —control parameters for microfluidics unit/manipulation parameters
A_{V}—amplitude of applied voltage
f_{V}—frequency of applied voltage
P_{V}—pulse duration of applied voltage
L_{V}—pulse delay of applied voltage
f_{D}—input rate of sample objects
Q_{C}—input flow rate of continuous phase
Q_{D}—input flow rate of dispersed phase
Q_{W}—withdrawal rate of continuous phase and dispersed phase

1. A method (500) of operating a microfluidic system (100, 200, 200′, 300), wherein the microfluidic system (100, 200, 200′, 300) comprises a manipulation zone (116) for manipulating sample objects (102A, 102B) flowing through the manipulation zone (116), a first detection zone (114) arranged upstream of or in the manipulation zone (116) and a second detection zone (118) arranged upstream of, in or downstream of the manipulation zone (116), the method (500) comprising:
determining a first count (N₁) that characterizes a number of sample objects (102A, 102B) flowing through the first detection zone (114);
manipulating sample objects (102A, 102B) in the manipulation zone (116);
determining a second count (N₂) that characterizes a number of sample objects (102A, 102B) flowing through the second detection zone (116); and
adjusting one or more manipulation parameters (x₁, x₂, y₁, y₂, . . . ) for manipulating the sample objects (102A, 102B) in the manipulation zone (116) based on the first count (N₁) and the second count (N₂).

2. The method (500) of claim 1, wherein the second detection zone (118) is arranged downstream of the first detection zone (114), the first count (N₁) is an input count (Nᵢₙ) that characterizes a number of non-manipulated sample objects (102A, 102B) flowing through the first detection zone (114) prior to manipulation in the manipulation zone (116), and the second count (N₂) is an output count (Nₒᵤₜ) that characterizes a number of manipulated sample objects (102A, 102B) flowing through the second detection zone (118) after manipulation in the manipulation zone (116).

3. The method (500) of claim 1 or 2, wherein the first count (N₁) and/or the second count (N₂) is determined by optical measurements, acoustic measurements and/or electrical measurements, in particular impedance measurements, on the sample objects (102A, 102B) flowing through the respective detection zone (114, 118).

4. The method (500) of any one of the preceding claims, wherein manipulating the sample objects (102A, 102B) in the manipulation zone (116) comprises deflecting the sample objects (102A, 102B) into one of a plurality of sorting channels (106A, 106B) based on a sorting control signal and the second count (N₂) characterizes a number of sample objects (102A, 102B) flowing along one of the sorting channels (106A, 106B).

5. The method (500) of any one of the preceding claims, wherein manipulating the sample objects (102A, 102B) in the manipulation zone (116) comprises merging two or more sample objects (102A, 102B) to a composite sample object.

6. The method (500) of any one of the preceding claims, wherein manipulating the sample objects (102A, 102B) in the manipulation zone (116) comprises generating a force on the sample objects (102A, 102B) by applying a voltage to an electrode (122) of the microfluidic system (100, 200, 200′, 300) and the one or more manipulation parameters (x₁, x₂, y₁, y₂, . . . ) comprise one or more of an amplitude (A_{V}) of the voltage, a frequency (f_{V}) of the voltage, a pulse duration (P_{V}) of the voltage, a pulse delay (L_{V}) of the voltage, and one or more flow rates (Q_{D}, Q_{C}, Q_{W}) of fluid flows in the microfluidic system (100, 200, 200′, 300).

7. The method (500) of claim 6, wherein adjusting the one or more manipulation parameters (x₁, x₂, y₁, y₂, . . . ) comprises determining an efficiency metric (η) of the manipulation of the sample objects (102A, 102B) based on the first count (N₁) and/or the second count (N₂) and optimizing the efficiency metric (η) by scanning one or more of the amplitude (A_{V}) of the voltage, the frequency (f_{V}) of the voltage, the pulse duration (P_{V}) of the voltage, the pulse delay (L_{V}) of the voltage, and the one or more flow rates (Q_{D}, Q_{C}, Q_{W}) of fluid flows in the microfluidic system (100, 200, 200′, 300) to determine a set of optimized manipulation parameters.

8. The method (500) of claim 7, wherein optimizing the efficiency metric (η) comprises:
scanning the pulse delay (L_{V}) at a constant pulse duration (P_{V}) and a constant amplitude (A_{V}) until the efficiency metric (η) meets an optimization criterion (ηₑ); and
if the optimization criterion (ηₑ) cannot be met by scanning the pulse delay (L_{V}) at the constant pulse duration (P_{V}) and the constant amplitude (A_{V}), scanning the pulse duration (P_{V}) at the constant amplitude (A_{V}),
in particular wherein optimizing the efficiency metric (η) further comprises scanning the amplitude (A_{V}) if the optimization criterion (ηₑ) cannot be met by scanning the pulse duration (P_{V}) at the constant amplitude (A_{V}).

9. The method (500) of claim 7 or 8, wherein the sample objects (102A, 102B) flow into the manipulation zone (116) at a constant input rate (f_{D}) and the method (500) comprises optimizing the efficiency metric (η) at two or more input rates (f_{D}) to determine respective sets of optimized manipulation parameters.

10. The method (500) of claim 9, wherein the optimization criterion (ηₑ) is a minimum manipulation efficiency or a maximum manipulation error and the method further comprises determining a maximum input rate at which the minimum manipulation efficiency or the maximum manipulation error, respectively, can be achieved.

11. The method (500) of any one of claims 7 to 10, further comprising determining a stability metric for the set of optimized manipulation parameters by determining the efficiency metric (η) for the set of optimized manipulation parameters again at one or more later points in time.

12. The method (500) of any one of the preceding claims, wherein the method (500) further comprises:
monitoring the first count (N₁) and the second count (N₂) continuously during operation of the microfluidic system (100, 200, 200′, 300); and
using the first and second counts (N₁, N₂) as inputs for a feedback control of the one or more manipulation parameters (x₁, x₂, y₁, y₂, . . . ) and/or for determining whether a failure has occurred during operation of the microfluidic system (100, 200, 200′, 300).

13. The method (500) of any one of the preceding claims, wherein the sample objects (102A, 102B) are microdroplets forming a dispersed phase in a continuous phase and the method (500) further comprises adjusting one or more of an input flow rate (Q_{D}) of the dispersed phase, an input flow rate (Q_{C}) of the continuous phase and a withdrawal rate (Q_{W}) of the dispersed and continuous phases based on the first count (N₁) and the second count (N₂).

14. The method (500) of any one of the preceding claims, further comprising determining one or more of a flow rate, a spacing, a size, and a velocity of the sample objects (102A, 102B) in the first detection zone (114) and/or in the second detection zone (118).

15. A microfluidic system (100, 200, 200′, 300) for manipulating sample objects (102A, 102B), wherein the microfluidic system (100, 200, 200′, 300) comprises:
a microfluidic channel (106, 106A-D) extending through a manipulation zone (116), a first detection zone (114) arranged upstream of or in the manipulation zone (116), and a second detection zone (118) arranged upstream of, in or downstream of the manipulation zone (116);
a first electrode assembly (120) arranged in the first detection zone (114) for performing electrical measurements on sample objects (102A, 102B) within the microfluidic channel (106, 106A-D);
means (112, 122, 304) for manipulating sample objects (102A, 102B) in the manipulation zone (116); and
a second electrode assembly (124) arranged in the second detection zone (118) for performing electrical measurements on sample objects (102A, 102B) within the microfluidic channel (106, 106A-D).

16. The microfluidic system (100, 200, 200′, 300) of claim 15, wherein the second detection zone (118) is arranged downstream of the first detection zone (114) and downstream of the means (112, 122, 304) for manipulating the sample objects (102A, 102B) in the manipulation zone (116).

17. The microfluidic system (200, 200′, 300) of claim 15 or 16, wherein the first electrode assembly (120) and/or the second electrode assembly (124) each comprise at least three electrodes that are in electrical contact with an inner volume of the microfluidic channel (106, 106A-D).

18. The microfluidic system (100, 200, 200′, 300) of any one of claims 15 to 17, wherein the microfluidic system (100, 200, 200′, 300) comprises a channel substrate (104B) in and/or on which the microfluidic channel (106, 106A-D) is formed and an electrode substrate (104A) bonded to the channel substrate (104B), wherein the first electrode assembly (120) and the second electrode assembly (124) are arranged on and/or in a surface of the electrode substrate (104A) facing the channel substrate (104B).

19. The microfluidic system (100, 200, 200′, 300) of any one of claims 15 to 18, wherein the means for manipulating the sample objects (102A, 102B) in the manipulation zone (116) comprise one or more of:
a manipulation electrode assembly (122) for manipulating the sample objects (102A, 102B) by electrophoresis, dielectrophoresis, and/or magnetophoresis;
an acoustic resonator for manipulating the sample objects (102A, 102B) by acoustophoresis;
a valve for manipulating the sample objects (102A, 102B) by adjusting a direction of flow and/or a flow velocity in the microfluidic channel (106, 106A-D); and
an illumination window for manipulating the sample objects (102A; 102B) by light-induced forces.

20. The microfluidic system (100, 200, 200′, 300) of any one of claims 15 to 19, wherein the means for manipulating the sample objects (102A, 102B) in the manipulation zone (116) comprise:
a sorting junction (112) at which the microfluidic channel (106) splits into a plurality of sorting channels (106A, 106B), wherein the second electrode assembly (124) is arranged along one (106A) of the sorting channels (106A, 106B); and/or
a merging junction (304) at which a plurality of sub-channels (106C, 106D) of the microfluidic channel (106) merge with each other, wherein the first electrode assembly (120) is arranged along one (106C) of the sub-channels (106C, 106D) between an inlet of the respective sub-channel (106C) and the merging junction (304) or along the microfluidic channel (106) between the merging junction (304) and the second electrode assembly (124).

21. A control system (400) for operating a microfluidic system (100, 200, 200′, 300), wherein the microfluidic system (100, 200, 200′, 300) comprises a manipulation zone (116) for manipulating sample objects (102A, 102B) flowing through the manipulation zone (116), a first detection zone (114) arranged upstream of or in the manipulation zone (116) and a second detection zone (118) arranged upstream of, in or downstream of the manipulation zone (116), the control system (400) comprising:
a mount (402) configured to receive the microfluidic system (100, 200, 200′, 300);
a measurement unit (404) configured to perform optical, acoustic, and/or electrical measurements in the first detection zone (114) and in the second detection zone (118) for detecting sample objects (102A, 102B) in the respective detection zone (114, 118);
a manipulation unit (406) configured to apply a force on sample objects (102A, 102B) in the manipulation zone (116) for manipulating the sample objects (102A, 102B); and
a controller (408),
wherein the controller (408) is configured to:
determine a first count (N₁) characterizing a number of sample objects (102A, 102B) flowing through the first detection zone (114) and a second count (N₂) characterizing a number of sample objects (102A, 102B) flowing through the second detection zone (118) based on the optical, acoustic, and/or electrical measurements performed by the measurement unit (404); and
adjust one or more manipulation parameters (x₁, x₂, y₁, y₂, . . . ) associated with the force applied by the manipulation unit (406) on the sample objects (102A, 102B) in the manipulation zone (116) based on the first count (N₁) and the second count (N₂).

22. The control system (400) of claim 21, wherein the second detection zone (118) is arranged downstream of the first detection zone (114), the first count (N₁) is an input count (Nᵢₙ) that characterizes a number of non-manipulated sample objects (102A, 102B) flowing through the first detection zone (114) prior to manipulation in the manipulation zone (116), and the second count (N₂) is an output count (Nₒᵤₜ) that characterizes a number of manipulated sample objects (102A, 102B) flowing through the second detection zone (118) after manipulation in the manipulation zone (116).

23. The control system (400) of claim 21 or 22, wherein the manipulation unit (406) is configured to apply the force by applying a voltage to an electrode (122) in the manipulation zone (116) and the one or more manipulation parameters (x₁, x₂, y₁, y₂, . . . ) comprise one or more of an amplitude (A_{V}) of the voltage, a frequency (f_{V}) of the voltage, a pulse duration (P_{V}) of the voltage, a pulse delay (L_{V}) of the voltage, and one or more flow rates (Q_{D}, Q_{C}, Q_{W}) of fluid flows in the microfluidic system (100, 200, 200′, 300).

24. The control system (400) of any one of claims 21 to 23, wherein the measurement unit (404) is configured to perform differential electrical measurements between two or more pairs of electrodes (120, 124) in one or both of the first detection zone (114) and the second detection zone (118).

25. The control system (400) of any one of claims 21 to 24, further comprising a sorting unit (410) that is configured to:
perform acoustic, optical and/or electrical measurements on sample objects (102A, 102B) in the microfluidic system (100, 200, 200′, 300);
classify the sample objects (102A, 102B) based on the acoustic, optical and/or electrical measurements; and
using the manipulation unit (406), guide the sample objects (102A, 102B) into one of a plurality of sorting channels (106A, 106B) in the manipulation zone (116) based on the classification.

26. The control system (400) of any one of claims 21 to 25, wherein the manipulation unit (406) is configured to manipulate the sample objects (102A, 102B) by merging two or more sample objects (102A, 102B) to a composite sample object.

27. The control system (400) of any one of claims 21 to 26, wherein the controller (408) is configured to execute a method (500) according to any one of claims 1 to 14.
